(19) United States
(12) Patent Application Publication
(10) Pub. No.: US 2003/0077289 A1
(43) Pub. Date: Apr. 24, 2003

(54) Use of cell-penetrating peptides to generate antitumor immunity

(76) Inventor: Rong-Fu Wang, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 10/077,555

(22) Filed: Feb. 15, 2002

Related U.S. Application Data
(60) Provisional application No. 60/268,687, filed on Feb. 15, 2001.

(51) Int. Cl. A61K 39/00

(52) U.S. Cl. 424/185.1; 424/277.1

(57) ABSTRACT

The present invention is directed to methods and compositions for enhancing an immune response in an animal to a disease by administering an immune effector cell comprising a cell penetrating peptide associated with an antigen for the disease. In a specific embodiment, the immune effector cell is a dendritic cell comprising a cell penetrating peptide associated with an antitumor antigen for cancer immunotherapy.

This application claims priority to U.S. Provisional Patent Application Serial No. 60/268,687, filed Feb. 15, 2001.

### FIELD OF INVENTION

The present invention is directed to the fields of immunology, cell biology, and cancer. Specifically, the present invention regards enhancement of immunity to disease. In specific embodiments, the present invention relates to methods and compositions for augmenting an immune response to a tumor in an animal.

### BACKGROUND OF THE INVENTION

T cells play an important role in immunological surveillance, such as against cancer cells as well as in tumor destruction. Successful identification of a number of tumor antigens from both human and murine melanomas has aided our understanding of the molecular basis of T cell-mediated antitumor immunity and set the stage for developing new strategies for antigen-specific cancer vaccines (Wang and Rosenberg, 1999; Boon and Vander Bruggen, 1996; Gilboa, 1999; Houghton et al., 2001). The majority of these antigens is nonmutated self-antigens and tends to elicit weak self-reactive T cell responses (Houghton et al., 2001). Immunotherapy for human cancer based on this growing list of target antigens thus far has met with only limited success (Dallal and Lotze, 2000). Thus, a major challenge in cancer vaccines is how to break self-tolerance and generate strong, long-lasting antitumor immunity through manipulation of both the antigen and delivery system.

One approach to improving peptide-based vaccines is to use dendritic cells (DCs) as an adjuvant (Schuler and Steinman, 1997; Banchereau and Steinman, 1998). Mature DCs pulsed with peptides have proven effective in enhancing antitumor immunity, although most studies used foreign antigens or peptides (OVA and βgal) as immune targets (Celluzzi et al., 1996; Paglia et al., 1996; Young and Inaba, 1996). Recently, both human and animal studies using mature DCs pulsed with tumor antigen-derived peptides showed some effect on increased T cell responses and inhibited tumor growth (Nestle et al., 1998; Thurner et al., 1999; Bellone et al., 2000; Schreurs et al., 2000), but compelling evidence showing that immunization with DC/peptides resulted in the complete tumor regression or therapeutic benefit is still lacking (Dallal and Lotze, 2000). It is currently unclear why immunization with DCs/peptide has not been as effective as originally anticipated. One possibility is that the half-life of MHC class I/peptide complexes on the DC surface is critical in determining T cell responses. Enhanced stability of MHC-peptide or MHC-peptide-TCR complexes on the cell surface by substituting favorable key peptide residues has correlated with improved T cell responses both in vitro and in vivo (Parkhurst et al., 1996; Rosenberg et al., 1998; Slansky et al., 2000).

Although mature DCs are more potent than immature DCs in priming and eliciting T cell responses (Labeur et al., 1999), they lose the ability to efficiently take up exogenous antigens (Banchereau and Steinman, 1998). As a result, peptide-pulsed DCs as vaccines have several limitations. Peptide degradation, rapid MHC class I turnover, and the disassociation of peptide from MHC class I molecules during the preparation and injection of mature DC pulsed with peptides may result in short half-lives of MHC class I/peptide complexes on the DC surface, leading to weak and transient T cell responses (Dallal and Lotze, 2000; Cella et al., 1997; Ludewig et al., 1999).

Thus, the present invention addresses a longfelt need in the art to provide effective methods and compositions for enhancing immunity in an animal utilizing immune effector cells, such as dendritic cells, wherein there is prolonged efficient presentation of a peptide to T cells, thereby resulting in the enhancement.

### SUMMARY OF THE INVENTION

To overcome deficiencies in the art, the present invention is directed to methods and compositions for intracellular delivery of a self-antigen peptide into cells with a cell-penetrating peptide (CPP). That is, the intracellular delivery of a self-peptide into mature DCs by a cell-penetrating peptide (CPP) could allow DCs to process and present the internalized peptides to T cells by newly synthesized MHC class I molecules for a long time. Several CPPs have been identified, from proteins, including the Tat protein of human immunodeficiency virus (HIV) (Frankel and Pabo, 1988), the VP22 protein of herpes simplex virus (Elliott and O'Hare, 1997; Phelan et al., 1998), and the fibroblast growth factor (Lin et al., 1995; Rojas et al., 1998). The Tat peptide and membrane-translocating sequence (MTS) have been used to transduce proteins into cells both in vitro and in vivo (Fawell et al., 1994; Kim et al., 1997; Schwarze et al., 1999; Lindgren et al., 2000; Caron et al., 2001). The use of these CPPs in delivering tumor antigens/peptides into DCs for generating potent immune responses against cancer has not heretofore been studied. Thus, this approach prolongs the efficient presentation of peptide to T cells, leading to the generation of potent antipeptide immunity, and the immune response is therefore enhanced.

Several cell-penetrating peptides have been identified from proteins, as described above, and the CPPs have the ability to deliver peptides or proteins in a variety of cell types in vitro and in vivo. However, it was not previously known whether the use of DC loaded with a CPP linked to a T cell peptide could generate potent immune responses against, for example, cancer. The present invention is a novel approach that generates a strong and long-lasting immune response.

The antigen utilized in the methods and compositions is directed against a specific disease, such as cancer. Other embodiments include autoimmune diseases and/or infectious diseases. Human TRP-2 and the murine homolog have been defined as two tissue-specific tumor antigens expressed on normal melanocytes and melanoma. Since HLA-A2 restricted T cell epitope from human TRP-2 is identical to that from murine TRP-2 recognized by Kb-restricted T cells, the use of murine TRP-2 as a self-antigen represents an ideal antigen to address questions important for immunotherapy of cancer in humans. However, dendritic cells (DC) pulsed with a TRP-2 self-antigen peptide failed to protect mice from tumor challenge. Surprisingly, intracellular delivery of a self-antigen peptide into DC using cell penetrating peptides (CPP) prolongs the efficient presentation of peptides to T cells, advantageously leading to the generation of a potent immune response, such as an antitumor immunity. Immunization of dendritic cells loaded with the TRP-2 peptide covalently linked to CPP completely protected mice from subsequent tumor challenge and significantly inhibited lung metastases in a three-day tumor model. Antibody depletion experiments indicated that CD8+T cells played an important role in inhibiting tumor growth and that CD4+T cells also played a role. In accordance with one aspect of the invention, intracellular delivery of CPP-linked self-antigen peptides represents a novel approach to immunotherapy research and treatment of cancer.

In an embodiment of the present invention, there is a composition comprising an immune effector cell and a cell penetrating peptide, wherein said cell penetrating peptide is associated with an antigen.

In another embodiment of the present invention, there is a composition comprising an immune effector cell and a cell penetrating peptide, wherein said cell penetrating peptide is associated with an antibody. In a specific embodiment, the antigen is a molecule comprising multiple T-cell peptides. In another specific embodiment, the multiple T-cell peptides are from either the same tumor antigen or different tumor antigens. In a further specific embodiment, the antigen comprises at least one MHC class I-restricted peptide, at least one MHC class II-restricted peptide, or at least one MHC class I-restricted peptide and at least one MHC class II-restricted peptide. In an additional specific embodiment, the immune effector cell is a mature dendritic cell, a B cell, a macrophage, or a fibroblast. In a further specific embodiment, the immune effector cell is a mature dendritic cell or a B cell. In a specific embodiment, the immune effector cell is a mature dendritic cell. In a specific embodiment, the antigen is a tumor antigen. In another specific embodiment, the tumor antigen is a peptide. In an additional specific embodiment, the tumor antigen is TRP2. In a further specific embodiment, the tumor antigen is one listed in Table 1, 2, 3, 4, or 5. In another specific embodiment, the cell penetrating peptide is CPP1, ANTP, Signal-peptide I, Signal-peptide II, PRES, Transportan, Amphiphilic model peptide, HSV VP22, peptide carrier, or CL22. In a further specific embodiment, the cell penetrating peptide is CPP1. In an additional specific embodiment, the association of the cell penetration peptide with the antigen is a covalent bond. In another specific embodiment, the antigen is housed within a vesicle in said immune system cell. In a further specific embodiment, the vesicle is an endosome.

In another embodiment of the present invention, there is a vaccine comprising an immune effector cell and a cell penetrating peptide, wherein said cell penetrating peptide is associated with an antigen; and a pharmaceutically acceptable carrier. In a specific embodiment, the immune effector cell is a mature dendritic cell, a B cell, a macrophage, or a fibroblast. In an additional specific embodiment, the immune effector cell is a mature dendritic cell or a B cell. In a further specific embodiment, the immune effector cell is a mature dendritic cell.

In an additional embodiment of the present invention, there is a method of enhancing immunity in an animal to a disease, comprising the step of administering to the animal a mature dendritic cell, wherein the cell comprises a cell penetrating peptide associated with an antigen to said disease, wherein following said administration, said animal is protected from said disease. In a specific embodiment, the animal comprises both CD4+ and CD8+T cells. In another specific embodiment, the dendritic cell is administered to the animal by injection. In a further specific embodiment, the injection is intravenously, intraperitoneally, or subcutaneously. In an additional specific embodiment, the animal is a mammal. In another specific embodiment, the mammal is a human.

In another embodiment of the present invention, there is a method of immunizing an animal, comprising administering to said animal at least once the vaccine comprising an immune effector cell and a cell penetrating peptide, wherein said cell penetrating peptide is associated with an antigen.

In another embodiment of the present invention, there is a method of treating a disease in an animal, comprising the step of administering to the animal an immune effector cell comprising a cell-penetrating peptide associated with an antigen for said disease; and a pharmaceutically acceptable carrier. In a specific embodiment, the immune effector cell is a mature dendritic cell, a B cell, a macrophage, or a fibroblast. In a specific embodiment, the immune effector cell is a mature dendritic cell or a B cell. In another specific embodiment, the immune effector cell is a mature dendritic cell. In a further specific embodiment, the cell penetrating peptide is CPP1, HIV Tat, VP22, MTS, or fibroblast growth factor. In an additional specific embodiment, the cell-penetrating peptide is CPP1. In another specific embodiment, the disease is cancer and wherein the antigen is a tumor antigen. In a further specific embodiment, the tumor antigen is TRP2. In a further specific embodiment, the tumor antigen is one listed in Table 1, 2, 3, 4, or 5. In an additional specific embodiment, the animal is further subjected to a cancer treatment, wherein the treatment is surgery, radiation, chemotherapy, or gene therapy. In another specific embodiment, the administration of the dendritic cell is prior to the cancer treatment. In another specific embodiment, the administration of the dendritic cell is subsequent to the cancer treatment. In an additional specific embodiment, the administration of the dendritic cell is concurrent with the cancer treatment.

In another embodiment of the present invention, there is a method of preparing a composition for a disease, comprising providing an immune effector cell; providing a cell penetrating peptide associated with an antigen for said disease; and introducing the cell penetrating peptide associated with the antigen to the immune effector cell, wherein said antigen enters into the cell. In a specific embodiment, the immune effector cell is a mature dendritic cell, B cell, macrophage, or fibroblast. In an additional specific embodiment, the immune effector cell is a mature dendritic cell. In an additional specific embodiment, the antigen is a tumor antigen, autoantigen, or viral antigen.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIGS. 1A through 1D demonstrate intracellular delivery of CPP1-peptides into dendritic cells and the prolonging of their ability to stimulate T cells. 1A) Peptide sequences for cell-penetrating peptide (CPP1), TRP-2 and control peptides. B) Fluorescence and phase-contrast microscopy of DC incubated with various peptide-FITC (40×magnification). DCs incubated with PBS served as a control. 1C) DCs incubated with CPP1-TRP2-FITC and viewed by phase-contrast or fluorescence microscopy at 60×magnification. Representative examples of DC/CPP1-TRP2-FITC are shown. The penetrating fluorescent peptide was localized in both the cytoplasm as well as the cell nucleus. 1D) T cell response to DCs loaded with each peptide (solid circle for CPP1-TRP2, open circle for TRP2, and open triangle for CPP1-βgal) at different time points after peptide pulsing. GM-CSF release from T cells was determined by ELISA.

FIGS. 2A and 2B show immunization of mice with mature DCs pulsed with CPP1-TRP2 generates protective immunity. 2A) Number of lung metastases in C57BL/6 mice after a single intravenous injection of DCs loaded with each indicated peptides, followed in two weeks by intravenous injection of B16 tumor cells. The experiments were repeated three times with similar results. 2B) Gross pathology of lungs from mice receiving each treatment.

FIGS. 3A and 3B are a comparative analysis of lung metastases and animal survival. 3A) Number of lung metastases in C57BL/6 mice after injection of mature, immature DCs and splenocytes pulsed with various peptides as indicated, and followed in two weeks by intravenous injection of B16 tumor cells. Mean numbers of lung metastases±SEM are presented. 3B) Mice were immunized with mature DCs loaded with various peptides twice in two-week interval. The immunized mice were then challenged with B16 tumor cells. Animal survival was monitored up to 60 days post tumor challenge.

FIG. 4 illustrates induction of CD8+T cell responses after vaccination. Recognition of target cells by T cells generated from splenocytes of mice immunized with DCs pulsed with the TRP2, CPP1-TRP2 or CPP1-βgal peptides. The splenocytes of two mice from each group were re-stimulated with the TRP2 peptide in vitro and T cells were tested against 293K^{b}, 293K^{b} pulsed with TRP2, B16 tumor or MHC class I-matched MC38 tumor cells. T cell activity was determined based on IFN-gamma release measured by ELISA.

FIGS. 5A through 5B illustrate a requirement of CD4+ and CD8+T cells for antitumor immunity. 5A) C57BL/6 mice were immunized with DC/CPP1-TRP2. Two weeks later, mice were treated with anti-CD4, anti-CD8 and control antibodies on the day before tumor challenge, and followed by three injections on day 1, 3 and 10 after tumor injection. Lung metastases counted from each group are presented. Mice immunized with DC/PBS and DC/CPP1-βgal were used as control groups for tumor injection and specificity. 5B) B6, CD4 KO and CD8 KO were immunized with DC/CPP1-TRP2, and then challenged with B16 tumor cells. B6 mice immunized with DC/PBS were used as a control. After 14 days of tumor challenge, the number of lung metastases was counted and plotted as mean numbers of lung metastases. Similar results were obtained three repeated experiments.

FIGS. 6A through 6B. Treatment of active B16 tumor with DCs pulsed with the CPP1-TRP2 peptide. 6A) Mice were intravenously injected with B16 tumor cells, and 3 days later were immunized with DCs loaded with the indicated peptides. Fourteen days after vaccination, lung metastases were counted in a blinded fashion. Mean numbers of lung metastases are presented in two separate experiments. The asterisk indicates a significant difference from each other groups (P<0.01) determined with the Wilcoxon Rank Sum test. Splenocytes pulsed with DCs/CPP1 did not reduce the number of lung metastasis. 6B) Photographs were taken of lungs from mice receiving each treatment in a separate experiment.

### DETAILED DESCRIPTION OF THE INVENTION

In keeping with long-standing patent law convention, the words “a” and “an” when used in the present specification in concert with the word comprising, including the claims, denote “one or more.”

The practice of the present invention will employ, unless otherwise indicated, conventional techniques of molecular biology, microbiology, recombinant DNA, and immunology, which are within the skill of the art. Such techniques are explained fully in the literature. See e.g., Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), ANIMAL CELL CULTURE (R. I. Freshney, Ed., 1987), the series METHODS IN ENZYMOLOGY (Academic Press, Inc.); GENE TRANSFER VECTORS FOR MAMMALIAN CELLS (J. M. Miller and M. P. Calos eds. 1987), HANDBOOK OF EXPERIMENTAL IMMUNOLOGY, (D. M. Weir and C. C. Blackwell, Eds.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987), CURRENT PROTOCOLS IN IMMUNOLOGY (J. E. Coligan, A. M. Kruisbeek, D. H. Margulies, E. M. Shevach and W. Strober, eds., 1991); ANNUAL REVIEW OF IMMUNOLOGY; as well as monographs in journals such as ADVANCES IN IMMUNOLOGY. All patents, patent applications, and publications mentioned herein, both supra and infra, are hereby incorporated herein by reference.

I. Definitions

The term “antigen” as used herein is defined as or any peptide involved in diseases such cancer, autoimmune and infectious diseases that can elicit an immune response. Some examples include tumor antigens, autoantigens, and viral antigens.

The term “tumor rejection antigen or tumor associated antigen” as used herein refers to an antigen capable of eliciting an immune response in an animal to a tumor. In another specific embodiment, the antigen is on the surface of malignant cells, is unique to the cancerous cells and is not present on their normal counterparts. In a specific embodiment, the anti-tumor antigen is a tumor-associated antigen, which is an antigen present on both normal and cancerous cells but ‘hidden’on normal cells, becoming ‘visible’when malignant, or overexpressed on the latter, as a product of cellular oncogenes.

The term “cell penetrating peptide” as used herein is defined as a peptide having the ability to transduce another peptide or protein into a cell in vitro and/or in vivo. In a specific embodiment the cell is an immune effector cell. In a further specific embodiment, the cell is a dendritic cell. In another specific embodiment, the cell penetrating peptide is CPP1, HIV Tat, VP22, MTS, or fibroblast growth factor.

The term “dendritic cell” refers to any member of a diverse population of morphologically similar cell types found in lymphoid or non-lymphoid tissues. These cells are characterized by their distinctive morphology, high levels of surface MHC-class II expression (Steinman et al., 1991). These cells can be isolated from a number of tissue sources, as is well known in the art. Furthermore, a skilled artisan recognizes that antigen-pulsed dendritic cells have traditionally been prepared in one of two ways: (1) small peptide fragments, known as antigenic peptides, are “pulsed” directly onto the outside of the APCs (Mehta-Damani et al., 1994); or (2) APCs are incubated with whole proteins or protein particles which are then ingested by the APCs. These proteins are digested into small peptide fragments by the APC and eventually carried to and presented on the APC surface (Cohen et al., 1994).

The cell surface of dendritic cells is unusual, with characteristic veil-like projections, and is characterized by having the cell surface markers CD1⁺, CD4⁺, CD86⁺, or HLA-DR⁺. Dendritic cells have a high capacity for sensitizing MHC-restricted T cells and are very effective at presenting antigens to T cells in situ, both self-antigens during T cell development and tolerance and foreign antigens during immunity.

Because of their effectiveness at antigen presentation, there is growing interest in using dendritic cells ex vivo as tumor or infectious disease vaccine adjuvants (see, for example, Romani et al., (1994)). The use of dendritic cells as immunostimulatory agents has been limited due to the low frequency of dendritic cells in peripheral blood, the limited accessibility of lymphoid organs and the dendritic cells'terminal state of differentiation. Dendritic cells originate from CD34+ bone marrow or peripheral blood progenitors and peripheral blood mononuclear cells, and the proliferation and maturation of dendritic cells can be enhanced by the cytokines GM-CSF (sargramostim, Leukine.RTM., Immunex Corporation, Seattle, Wash.), TNF-α, c-kit ligand (also known as stem cell factor (SCF), steel factor (SF), or mast cell growth factor (MGF)) and interleukin-4. Flt3-L has been found to stimulate the generation of large numbers of functionally mature dendritic cells, both in vivo and in vitro (U.S. Ser. No. 08/539,142, filed Oct. 4, 1995).

The term “immune effector cell” as used herein is defined as any cell which is capable of eliciting a T cell response in an animal and which is capable of uptake, and preferably also presentation, of an antigen, wherein the antigen is delivered to the cell via a cell penetrating peptide.

The term “mature dendritic cell” as used herein is defined as dendritic cells that express high level of MHC class II, CD80 (B7.1) and CD86 (B7.2) molecules, while immature dendritic cells express low levels of MHC class II, CD80 (B7.1) and CD86 (B7.2) molecules but have a great capacity of uptaking antigens.

The term “peptide” as used herein is defined as a small molecule of covalently bonded amino acids.

The term “self-antigen peptide” as used herein is defined as a nonmutated peptide derived from a tumor antigen expressed in both cancer and tissue-specific normal cells. In a specific embodiment, the peptide is about 9 to 15 amino acids in length.

II. The Present Invention

Vaccination with dendritic cells (DCs) pulsed with antigenic peptides derived from various tumor antigens has great potential in cancer treatment, but thus far has showed only limited success. The present invention is a novel strategy to enhance T cell responses by prolonging presentation of an MHC class I-restricted self-peptide on DCs through the use of a cell penetrating peptide (CPP). In a specific embodiment, DCs loaded with a peptide derived from tyrosinase-related protein 2 (TRP2) covalently linked to a CPP1 sequence retained full capacity to stimulate T cells while the ability of DCs pulsed with the TRP2 peptide alone dramatically diminished within 24 h. Immunization with DCs with the internalized peptide completely protected mice from subsequent tumor challenge, and significantly inhibited lung metastases in a 3-day tumor model, but DCs pulsed with TRP2 alone were ineffective. In addition, we demonstrated that both CD4+ and CD8+T cells were required for potent antitumor immunity. These results suggest that this novel approach may be generally applicable to enhance the efficacy of DC-based peptide vaccines against cancer, and may extend to the therapeutic treatment of other diseases as well.

In a specific embodiment of the present invention, a TRP2 peptide (SVYDFFVWL; SEQ 1D NO:2) and a 12-mer CPP1 (AAVLLPVLLAAP; SEQ 1D NO:1) (also called MTS) (Rojas et al., 1998) are utilized in animal tumor models. The HLA-A2 restricted T cell epitope from human TRP2 is identical to one identified in murine TRP2 (Wang et al., 1996; Bloom et al., 1997; Parkhurst et al., 1998). CPP1 can efficiently deliver the TRP2 peptide into mature DCs and retains the full capacity of DCs to present MHC-peptide complexes to antigen-specific T cells for a prolonged period of time. Immunization of mice with DCs bearing a TRP2 peptide linked to CPP1 resulted in complete protection against B16 tumor as well as in inhibition of the established tumor. Both CD4+ and CD8+T cells were required for generating antitumor immunity.

III. Cell Penetrating Peptides

The present invention utilizes cell penetrating peptides in the methods and compositions. The cell penetrating peptide facilitates intracellular delivery of the desired antigen into the immune effector cell. The technology of protein transduction is known in the art, such as is described in Rojas et al. (1998) and Schwarze et al. (1999), both incorporated by reference herein.

Several cell-penetrating peptides have been identified from proteins including TAT from human immunodeficiency virus (HIV) 18 19, and fibroblast growth factor (Ludewig et al., 1999; Frankel and Pabo, 1988). The CPPs have the ability to deliver peptides or proteins in a variety of cell types, in vitro and in vivo.

Some examples of cell penetrating peptides include:

| | | | |
|---|---|---|---|
| 1. | AAVLLPVLLAAP | (CPP1) | (SEQ ID NO:1) |
| 2. | RQIKIWFQNRRMKWKK | (ANTP) | (SEQ ID NO:3) |
| 3. | YGRKKRRQRRR | (HIV Tat 47-57) | (SEQ ID NO:4) |
| 4. | YARAAARQARA | (TAT-PTD-4) | (SEQ ID NO:5) |
| 5. | YARAARRAARR | (TAT-PTD-5) | (SEQ ID NO:6) |
| 6. | AAVALLPAVLLALLAP | (Signal-peptide I) | (SEQ ID NO:7) |
| 7. | GALFLGWLGAAGSTMGAWSQPKKKRKV | (Signal-peptide II) | (SEQ ID NO:8) |
| 8. | PLSSIFSRIGDP | (PRES) | (SEQ ID NO:9) |
| 9. | GWTLNSAGYLLKINLKALAALAKKIL | (Transportan) | (SEQ ID NO:10) |
| 10. | KLALKLALKALKAALKLA | (Amphiphilic model peptide) | (SEQ ID NO:11) |
| 11. | DAATATRGRSAASRPTERPRAPARSASRPRRPVE | (HSV VP22) | (SEQ ID NO:12) |
| 12. | KETWWETWWTEWSQPKKKRKV | (peptide carrier) | (SEQ ID NO:13) |
| 13. | KKKKKKGGFLGFWRGENGRKTRSAYERMCNILKGK | (CL22) | (SEQ ID NO:14) |

IV. Tumor Antigens

In the context of the present invention, “tumor antigen” refers to antigens that are common to specific tumor types. The tumor antigen of the present invention form part of, or are derived from, cancers including but not limited to primary or metastatic melanoma, thymoma, lymphoma, sarcoma, lung cancer, liver cancer, non-Hodgkin's lymphoma, Hodgkins lymphoma, leukemias, uterine cancer, cervical cancer, bladder cancer, kidney cancer and adenocarcinomas such as breast cancer, prostate cancer, ovarian cancer, pancreatic cancer, and the like. In one embodiment, the tumor antigen of the present invention comprises one or more antigenic cancer epitopes immunologically recognized by tumor infiltrating lymphocytes (TIL) derived from a cancer tumor of a mammal.

Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD 19, CD20, idiotype) have been used as targets for passive immunotherapy with monoclonal antibodies with limited success.

Thus, examples of tissue-specific tumor antigens include, but are not limited to prostatic acid phosphatase (PAP; associated with prostatic tumors), Melan-A/MART-1 (associated with melanoma; Coulie et al., 1994, J. Exp. Med. 180:35, Hawakami et al., 1994, PNAS 91:3515, Bakker et al., 1994, J. Exp. Med. 179:1005), tyrosinase/albino (associated with melanoma; Kawakami et al., 1994, J. Exp. Med.), and CD19, CD20 and CD37 (associated with lymphoma).

Likewise, oncogene product peptide antigens have been identified that are common to specific tumor types. These polypeptides will find use in the polypeptide complexes of the present invention as reagents that can be used generally to stimulate T-cell responses effective to react with tumors bearing such antigens. oncogene product peptide antigens include but are not limited to HER-2/neu (Beckmann et al., 1992, Eur. J. Cancer 28:322) associated with human breast and gynecological cancers, carcinoembryonic antigen (CEA) associated with cancer of the pancreas.

The tumor antigen and the antigenic cancer epitopes thereof may be purified and isolated from natural sources such as from primary clinical isolates, cell lines and the like. The cancer peptides and their antigenic epitopes may also be obtained by chemical synthesis or by recombinant DNA techniques known in the arts. Techniques for chemical synthesis are described in Steward et al. (1969); Bodansky et al. (1976); Meienhofer (1983); and Schroder et al. (1965).

Furthermore, as described in Renkvist et al. (2001), there are numerous antigens known in the art. The following tables describe T cell-defined epitopes encoded by tumor antigens, and only those tumor antigens recognized by T cells (either cytotoxic CD8+ or helper CD4+) are listed. Although analogs or artificially modified epitopes are not listed, a skilled artisan recognizes how to obtain or generate them by standard means in the art. Other antigens, identified by antibodies and as detected by the Serex technology (see Sahin et al. (1997) and Chen et al. (2000)), are identifed in the database of the Ludwig Institute for Cancer Research, which a skilled artisan can easily find on the World Wide Web.

The following table is from Renkvist et al. (2001).

TABLE 1
Class I HLA-restricted cancer/testis antigens. All these antigens were
found to be expressed by normal spermatocytes and/or spermatogonia
of testis. Occasionally MAGE-3, MAGE-4 and the GAGE genes were
found to be expressed also in placenta (De Backer et al., 1999;
Cox et al., 1994). The NY-ESO-1 antigen was found to be expressed
in normal ovary cells (Chen et al., 1997).

| | HLA | Peptide | | |
|---|---|---|---|---|
| Gene | allele | epitope | Authors | Tissue distribution among tumors^{a} |
| MAGE-A1 | A1 | EADPTGH | Traversari et al. 1992 | Melanoma, breast carcinoma, |
| MAGE-A1 | A3 | SY | Chaux et al. 1999a | SCLC (De Plaen et al., 1999); De |
| MAGE-A1 | A24 | SLFRAVI | Fujie et al. 1999 | Smet et al., 1994, van der Bruggen |
| MAGE-A1 | A28 | TK | Chaux et al. 1999a | et al., 1994a) - sarcoma, NSCLC |
| | | NYKHCFP | | (De Plaen et al., 1999), De Smet et |
| | | EI | | al., 1994) - thyroid medullary |
| | | EVYDGR | | carconoma (van der Bruggen et al., |
| | | EHSA | | 1994a) - colon carcinoma (De |
| | | | | Plaen et al., 1999) - laryngeal |
| | | | | tumors (De Smet et al., 1994) |
| MAGE-A1, | B37 | REPVTKA | Tanzarella et al. 1999 | Melanoma, colon and breast |
| -A2, -A3, | | EML | | carcinomas, SCLC (De Plaen et |
| -A6 | | | | al., 1999, De Smet et al., 1994, |
| | | | | van der Bruggen et al., 1994a) - |
| | | | | sarcoma, NSCLC (De Plaen et al., |
| | | | | 1999, De Smet et al., 1994- |
| | | | | thyroid medullary carcinoma, H/N |
| | | | | tumors, bronchial SCC (van der |
| | | | | Bruggen et al., 1994a) - laryngeal |
| | | | | tumors (De Smet et al., 1994) - |
| | | | | leukemias (De Plaen et al., 1994) |
| MAGE-A1 | B53 | DPARYEF | Chaux et al. 1999a | Melanoma, breast carcinoma, |
| MAGE-A1 | Cw2 | LW | Chaux et al. 1999a | SCLC (De Plaen et al., 1999, De |
| MAGE-A1 | Cw3 | SAFPTTIN | Chaux et al. 1999a | Smet et al., 1994, Van den Eynde |
| MAGE-A1 | Cw16 | F | van der Bruggen et al. | et al., 1999) - sarcoma, colon |
| | | SAYGEPR | 1994b | carcinoma, NSCLC (De Plaen et |
| | | KL | | al., 1999, De Smet et al., 1994) - |
| | | SAYGEPR | | thyroid medullary carcinoma (van |
| | | KL | | der Bruggen et al., 1994a) |
| MAGE-A2 | A2 | KMVELV | Visseren et al. 1997 | Melanoma, colon and breast |
| MAGE-A2 | A2 | HFL | Visseren et al. 1997 | carcinomas, SCLC (De Plaen et |
| MAGE-A2 | A24 | YLQVFGI | Tahara et al. 1999 | al., 1999, De Smet et al., 1994, |
| | | EV | | van der Bruggen et al., 1994a) - |
| | | EYLQLVF | | sarcoma, NSCLC (De Plaen et al., |
| | | GI | | 1999, De Smet et al., 1994) |
| | | | | thyroid medullary carcinoma (van |
| | | | | der Bruggen et al., 1994a) - |
| | | | | laryngeal tumors (Lurquin et al., |
| | | | | 1997) - leukemias (De Plaen et al., |
| | | | | 1999) |
| MAGE-A3 | A1 | EADIPIGH | Gaugler et al. 1994 | Melanoma, colon and breast |
| MAGE-A3 | A2 | LY | van der Bruggen et al. | carcinomas (De Plaen et al., 1999, |
| MAGE-A3 | A24 | FLWGPR | 1994a | van der Bruggen et al., 1994a) - |
| MAGE-A3 | A24 | ALV | Oiso et al. 1999 [ | H/N tumors (Chen et al., 1997) - |
| MAGE-A3 | B44 | TFPDLES | Tanaka et al. 1997 | bronchial SCC, thyroid medullary |
| | | EF | Hermann et al. 1996 | and bladder carcinoma, sarcomas, |
| | | IMPKAGL | Fleischhauer et | SCLC, NSCLC, (van der Bruggen |
| | | LI | al. 1996 | et al., 1994a) - leukemias (De |
| | | MEVDPIG | | Smet et al., 1994) |
| | | HLY | | |
| MAGE-A3 | B52 | WQYFFP | Russo et al. 2000 | |
| | | VIF | | |
| MAGE-A4 | A2 | GVYDGR | Duffour et al. 1999 | Melanoma, NSCLC, sarcomas, |
| | | EHTV | | esophageal, colon and breast |
| | | | | carcinomas (De Plaen et al., 1999) |
| MAGE-A6 | A34 | MVKISGG | Zorn and Hercent, | Melanoma, NSCLC, colon |
| | | PR | 1999b | carcinoma, leukemias (De Plaen et |
| | | | | al., 1999) |
| MAGE- | A2 | GLYDGM | Huang et al. 1999 | Not defined |
| A10 | | EHL | | |
| MAGE- | Cw7 | VRIGHLY | Panelli et at 2000 | Melanoma, myeloma, brain |
| A12 | | IL | Heidecker et al. 2000 | tumors, sarcoma, leukemias, |
| | | | | SCLC, NSCLC, H/N tumors, |
| | | | | bladder, lung, esophageal, breast, |
| | | | | prostate and colorectal carcinoma |
| | | | | (De Plaen et al., 1994) |
| BAGE | Cw16 | AARAVFL | Boer et al. 1995 | Melanoma, bladder and mammary |
| | | AL | | carcinomas, H/N SCC, NSCLC, |
| | | | | sarcoma |
| DAM-6, | A2 | FLWGPR | Fleischhauer et al 1998 | Melanoma, skin tumors, mammary |
| -10 | | AYA | | and ovarian carcinomas (Lurquin |
| | | | | et al., 1997) - lung carcinoma |
| | | | | (Dabovic et al., 1995; Lurquin et |
| | | | | al., 1997) - seminomas (Dabovic |
| | | | | et al., 1995) |
| GAGE-1, -2, | Cw6 | YRPRPRR | Van den Eynde et | Melanoma, sarcoma, NSCLC, |
| -8 | | Y | al. 1995 | SCLC, mesothelioma, sarcoma, |
| | | | De Backer et al. 1999 | seminoma, leukemias, lymphomas, |
| | | | | H/N tumors, bladder, esophageal, |
| | | | | mammary, colon, prostate |
| | | | | carcinomas |
| GAGE-1, | A29 | YYWPRP | De Backer et al 1999 | Melanomas, H/N tumors, |
| -4, -5, | | RRY | | leukemias, esophageal, lung and |
| -6, -7B | | | | bladder carcinomas |
| NA88-A | B13 | MTQGQH | Moreau-Aubrey, et al. | Melanoma |
| | | FLQKV | 2000 | |
| NY-ESO-1 | A2 | SLLMWIT | Jäger et al. 1998 | Melanoma, sarcoma, B- |
| NY-ESO- | A2 | QCFL | Jäger et al. 1998 | lymphomas, hepatoma, H/N |
| 1a | A2 | SLLMWIT | Jäger et al. 1998 | tumors, bladder, lung, prostate, |
| (CAG-3) | A31 | QC | Wang et al. 1998b | ovarian, thyroid and breast |
| | | QLSLLM | | carcinoma (Chen et al., 1997) |
| | | WIT | | |
| | | ASGPGGG | | |
| | | APR | | |

^{a}Tissue distribution among tumors as described in the given references when different from the paper first reporting the sequence of the epitope.

TABLE 2
Class I HLA-restricted melanocyte differentiation antigens. These antigens
can only be expressed in normal and neoplastic cells of the same lineage (namely
melanocytes, skin, retina, peripheral ganglia) or in normal cells of the prostate gland.

| Gene | HLA allele | Peptide epitope | Authors |
|---|---|---|---|
| MART- | A2 | AAGIGILTV | Coulie et al. 1994 |
| 1/Melan-A^{a} | | | Kawakami et al. 1994a |
| | A2 | EAAGIGILTV | Schneider et al. 1998 |
| | A2 | ILTVILGVL | Castelli et al. 1995 |
| | B45 | AEEAAGIGIL | Schneider et al. 1998 |
| | B45 | AEEAAGIGILT | Schneider et al. 1998 |
| MCIR | A2 | TILLGIFFL | Salazar-Onfray et al. 1997 |
| | A2 | FLALIICNA | Salazar-Onfray et al. 1997 |
| Gp100 | A2 | KTWGQYWQV | Bakker et al. 1995 |
| | A2 | AMLGTHTMEV | Tsai et al. 1997 |
| | A2 | MLGTHTMEV | Tsai et al. 1997 |
| | A2 | SLADTNSLAV | Tsai et al. 1997 |
| | A2 | ITDQVPFSV | Kawakami et al. 1995 |
| | A2 | LLDGTATLRL | Kawakami et al. 1994b |
| | A2 | YLEPGPVTA | Cox et al. 1994 |
| | A2 | VLYRYGSFSV | Kawakami et al. 1995 |
| | A2 | RLMKQDFSV | Kawakami et al. 1998 |
| | A2 | FLPRIFCSC | Kawakami et al. 1998 |
| | A3 | LIYRRRLMK | Kawakami et al. 1998 |
| | A3 | ALNFPGSQK | Kawashima et al. 1998 |
| | A3 | SLIYRRRLMK | Kawashima et al. 1998 |
| | A3 | ALLAVGATK | Skipper et al. 1996 |
| | A24 | VYFFLPDHL | Robbins et al. 1997 |
| | Cw8 | SNDGPTLI | Castelli et al. 1999 |
| PSA | A1 | VSHSFPHPLY | Corman et al. 1998 |
| | A2 | FLTPKKLQCV | Correale et al. 1997 |
| | A2 | VISNDVCAQV | Correale et al. 1997 |
| PSM | A1 | HSTNGVTRIY | Corman et al. 1998 |
| Tyrosinase | A1 | KCDICTDEY | Kittlesen et al. 1998 |
| | A1 | SSYVIPIGTY | Kawakami et al. 1998 |
| | A2 | YMDGTMSQV | Wölfel et al. 1994 |
| | A2 | MLLAVLYCL | Wölfel et al. 1994 |
| | A24 | AFLPWHRLF | Kang et al. 1995 |
| | B44 | SETWRDIDF | Brichard et al. 1996 |
| TRP-1 (or | A31 | MSLQRQFLR | Wang et al. 1996b |
| gp 75) | | | |
| TRP-2 | A2 | SVYDFFVWL | Parkhurst et al. 1998 |
| | A2 | TLDSQVMSL | Noppen et al. 2000 |
| | A31 | LLGPGRPYR | Wang et al. 1996a |
| | A33 | LLGPGRPYR | Wang et al. 1998a |
| | Cw8 | ANDPIFVVL | Castelli et al. 1999 |

^{a}Two different groups simultaneously discovered this gene and gave it two different names, MART-1 and Melan-A respectively

The following table is from Renkvist et al. (2001).

The following table is from Renkvist et al. (2001).

TABLE 3
Class I HLA-restricted widely expressed antigens

| | | | Tissue distribution | | |
|---|---|---|---|---|---|
| Gene | HLA | Peptide epitope | Tumors | Normal tissues | Reference |
| ART-4 | A24 | AFLRHAAL | SCC, SCLC, H/N | Testis, placenta, | Kawano et al. |
| | | DYPSLSATDI | tumors, leukemia, | fetal liver | 2000 |
| | | | lung, esophageal, | | |
| | | | gastric, cervical, | | |
| | | | endometrial, | | |
| | | | ovarian and breast | | |
| | | | carcinomas | | |
| CAMEL | A2 | MLMAQEALAFL | Melanoma | Testis, placenta, | Aarnoudse et |
| | | | | heart, skeletal | al. 1999 |
| | | | | muscle, pancreas | |
| CEA | A2 | YLSGANLNL | Melanoma | Testis, placenta, | Tsang et al. |
| | | (CAP-1)^{a} | | heart, skeletal | 1995 |
| | | | | muscle, pancreas | |
| CEA | A3 | HLFGYSWYK | Colon, rectum, | Gastrointestinal | Kawashima et |
| | | | pancreas, gastric, | embryonic tissue | al. 1999 |
| | | | breast and lung | | |
| | | | carcinomas | | |
| Cyp-B | A24 | KFHRVIKDF | Lung adenocarcin | Ubiquitously | Gomi et al. |
| | | DFMIQGGDF | oma, T cell | expressed in | 1999 |
| | | | leukemia, | normal tissues | |
| | | | lymphosarcoma - | | |
| | | | bladder, ovarian, | | |
| | | | uterine and | | |
| | | | esophagela SCC, | | |
| HER2/neu | A2 | KIFGSLAFL | Melanoma - | Epithelial cells | Risk et al. |
| | | | ovarian and breast | | 1995 |
| | | | carcinomas | | |
| HER2/neu | A2 | IISAVVGIL | Melanoma, ovarian, | Epithelial cells | Peoples et al. |
| | | | pancreatic | | 1995 |
| | | | (Pieper et al., | | |
| | | | 1999)^{b} and breast | | |
| | | | carcinomas | | |
| HER2/neu | A2 | RLLQETELV | Melanoma, | Epithelial cells | Kono et al. |
| | | | ovarian, gastric, | | 1998 |
| | | | pancreatic (Pieper | | |
| | | | et al., 1999) and | | |
| | | | breast carcinomas | | |
| HER2/neu | A2 | VVLGVVFGI | Melanoma, | Epithelial cells | Rongcun et al. |
| | | ILHNGAYSL | ovarian, gastric, | | 1999 |
| | | YMIMVKCWMI | pancreatic (Pieper | | |
| | | | et al., 1999) and | | |
| | | | breast carcimonas | | |
| HER2/neu | A3 | VLRENTSPK | Melanoma, | Epithelial cells | Kawashima et |
| | | | ovarian, gastric, | | al. 1999 |
| | | | pancreatic (Pieper | | |
| | | | et al., 1999) and | | |
| | | | breast carcinomas | | |
| HTERT^{c} | A2 | ILAKFLHWL | Lung, and ovarian | Hematopoietic | Vonderheide |
| | | | carcinomas - | stem cells and | et al. 1999 |
| | | | multiple myeloma, | progenitors; | |
| | | | melanoma, | germinal center | |
| | | | sarcoma, acute | cells; basal | |
| | | | leukemias, non- | keratinocytes; | |
| | | | Hodgkin's | gonadal cells; | |
| | | | lymphomas | certain | |
| | | | | proliferating | |
| | | | | epithelial cells | |
| HTRT^{c} | A2 | ILAKFLHWL | Lung, prostate and | Circulating B | Minev et al. |
| | | RLVDDFLLV | ovarian | cells; germinal | 2000 |
| | | | carcinomas, | center B cells; | |
| | | | multiple myeloma, | thymocytes; | |
| | | | melanoma, | CD34+ progeni- | |
| | | | sarcoma, acute | tor hemopoietic | |
| | | | leukemias, non- | cells | |
| | | | Hodgkin's | | |
| | | | lymphomas | | |
| iCE | B7 | SPRWWPTCL | RCC | Kidney, colon, | Ronsin et al. |
| | | | | small intestine, | 1999 |
| | | | | liver, heart, | |
| | | | | pituitary gland, | |
| | | | | adrenal gland, | |
| | | | | prostate, stomach | |
| MUCI | A11 | STAPPAHGV | Breast and ovarian | None^{d} | Domenech et |
| | | | carcinomas, | | al. 1995 |
| | | | multiple myeloma, | | |
| | | | B-cell carcinoma, | | |
| | | | multiple myeloma | | |
| MUCI | A2 | STAPPVHNV | Breast and ovarian | None^{d} | Brossart et al. |
| | | | carcinoma, | | 1999 |
| | | | multiple myeloma, | | |
| | | | B-cell lymphoma | | |
| MUC2 | A2 | LLNQLQVNL | Ovary, pancreas | Colon, small | Bohm et al. |
| | | MLWGWREHV | and breast | intestine, | 1998 |
| | | | mucinous tumors, | bronchus, cervix | |
| | | | colon carcinoma | and gall bladder | |
| | | | of non-mucinous | | |
| | | | type | | |
| PRAME | A24 | LYVDSLFFL | Melanoma, H/N | Testis, | Ikeda et al. |
| | | | and lung SCC, | endometrium, | 1997 |
| | | | NSCLC (van | ovary, adrenals, | |
| | | | Baren et al., | kidney, brain, | |
| | | | 1998), RCC, | skin | |
| | | | adenocarcinoma, | | |
| | | | sarcoma, | | |
| | | | leukemias | | |
| P15 | A24 | AYGLDFYIL | Melanoma | Testis, spleen, | Robbins et al |
| | | | | thymus, liver, | 1995 |
| | | | | kidney, adrenal | |
| | | | | tissue, lung | |
| | | | | tissue, retinal | |
| | | | | tissue | |
| RU1 | B51 | VPYGSFKHV | Melanoma, renal | Testis, kidney, | Morel et al. |
| | | | and bladder | heart, skin, brain, | 2000 |
| | | | carcinomas | ovary, liver, lung, | |
| | | | | lymphocytes, | |
| | | | | thymus, | |
| | | | | fibroblasts | |
| RU2 | B7 | LPRWPPPQL | Melanoma, | Testis, kidney, | Van den |
| | | | sarcomas | liver, urinary | Eynde et al. |
| | | | leukemia - brain, | bladder | 1999 |
| | | | esophageal and | | |
| | | | H/N tumors - | | |
| | | | renal, colon, | | |
| | | | thyroid, | | |
| | | | mammary, | | |
| | | | bladder, prostatic | | |
| | | | and lung | | |
| | | | carcinomas | | |
| SART-1 | A24 | EYRGFTQDF | Esophageal, H/N | Testis, fetal liver | Kikuchi et al. |
| | | | and lung SCC - | | 1999 |
| | | | adenocarcinoma, | | |
| | | | uterine cancer | | |
| SART-1 | A*2601 | KGSGKMKTE | Esophageal, H/N | Testis, fetal liver | Shichijo et al. |
| | | | and lung SCC, | | 1998 |
| | | | adenocarcinoma, | | |
| | | | uterine cancer | | |
| SART-3 | A24 | VYDYNCHVDL | H/N, esophageal | Lymphoid cells, | Yang et al |
| | | AYIDFEMKI | and lung SCC, | fibroblasts, testis, | 1999 |
| | | | adenocarcinoma, | fetal liver | |
| | | | leukemia, | | |
| | | | melanoma | | |
| WT1 | A2 | RMFPNAPYL | Gastric, colon, | Kidney, ovary, | Oka et al. |
| | | | lung, breast, | testis, spleen | 2000 |
| | | | ovary, uterine, | | |
| | | | thyroid and | | |
| | | | hepatocellular | | |
| | | | carcinomas - | | |
| | | | leukemia | | |
| | | | (including AML, | | |
| | | | ALL and CML) | | |

^{a}CAP-1 is an alternative name of this peptide
^{b}Tissue distribution among tumors as described in the given references when different from the paper first reporting the sequence of the epitope
^{c}Telomerase is expressed in most human tumors: those listed were shown to be susceptible to lysis by cytotoxic T lymphocytes
^{d}All epithelial tissues express mucin-like hyperglycosylated molecules

The following table is from Renkvist et al. (2001).

TABLE 4
CLASS I HLA-RESTRUCTED TUMOR-SPECIFIC ANTIGENS, INCLUDING BOTH
UNIQUE (CDK-4, MUM-2, β-CATENIN, HLA-A2-R170I, ELF2 M,
MYOSIN-M, CASPASE-8, KIAA0205, HSP70-2M) AND SHARED (CAMEL,
TRP-2/INT2, GNT-V 250, ANTIGENS

| | | | Tissue distribution | | |
|---|---|---|---|---|---|
| | HLA | | | Normal | |
| Gene | allele | Peptide epitope | Tumors | tissues | Reference |
| AFP | A2 | GVALQTMKQ | Hepatocellular | Fetal liver | Butterfield et al. |
| | | | carcinoma | | 1999 |
| β-Catenin/m | A24 | SYLDSGIHF | Melanoma | None | Robbins et al. 1996 |
| Caspase-8/m | A2 | FPSDSWCYF | H/N tumors | None | Mandruzzato et al. |
| | | | | | 1997 |
| CDK-4/m | A2 | ACDPHSGHFV | Melanoma | None | Wõfel et al. 1995 |
| ELF2 M | A68 | ETVSEQSNV | Lung SCC | None | Hogan et al. 1998 |
| GnT-V | A2 | VLPDVFIRC(V)^{a} | Melanoma, brain tumors, | Breast and | Guilloux et al. |
| | | | sarcoma | brain (low | 1996 |
| | | | | expression) | |
| G250 | A2 | HLSTAFARV | RCC, colon, ovarian and | None | Vissers et al. 1991 |
| | | | cervical carcinomas | | |
| HSP70-2M | A2 | SLFEGIDIY | RCC, melanoma, | None | Gaudin et al. 1999 |
| | | | neuroblastoma | | |
| HA-A*0201- | A2 | CVEWLRIYLE | RCC | None | Br\overline{a}ndle et al. 1996 |
| R1701 | | NGK | | | |
| HST-2 | A31 | YSWMDISCWI | Gastric signet cell | None | Suzuki et al. 1999 |
| | | | carcinoma | | |
| KIAA0205 | B44*03 | AEPINIQTV | Bladder cancer | None | Gueguen et al. |
| | | | | | 1998 |
| MUM-1 | B44 | EEKLIVVLF | Melanoma | None | Coulie et al. 1995 |
| MUM-2 | B44 | SELFRSGLDY | Melanoma | None | Coulie et al. 1995 |
| MUM-2 | Cw6 | FRSGLDSYV | Melanoma | None | Chiari et al. 1999 |
| MUM-3 | A28 | EAFIQPITR | Melanoma | None | Baurain et al. 2000 |
| Myasin/m | A3 | KINKNPKYK | Melanoma | None | Zorn and Hercend, |
| | | | | | 1999a |
| RAGE | B7 | SPSSNRIRNT | Melanoma, sarcomas, | Retina | Gaugler et al. 1996 |
| | | | mesotheliomas, H/N | only | |
| | | | tumors, bladder, renal, | | |
| | | | colon and mammary | | |
| | | | carcinomas | | |
| SART-2 | A24 | DYSARWNEI | H/N and lung SCC, lung | None | Nakao et al. 2000 |
| | | | adenocarcinoma, RCC, | | |
| | | | melanoma, brain tumors, | | |
| | | | esophageal and uterine | | |
| | | | cancers | | |
| TRP-2/INT2 | A68 | AYDFLYNYL | Melanoma | None | Lupetti et al. 1998 |
| | | SYTRLFLIL | | | |
| | | EVISCKLIKR | | | |
| 707-AP | A2 | RVAALARDA | Melanoma | None^{b} | Morioka et al. |
| | | | | | 1995 |

^{a}VLPDVFIRC(V) = nonamer and decamer peptides are both recognized by CTLs
^{b}This antigen is not expressed in normal cells but, as the tissue of the testis was not tested, it will not become clear to which category the antigen may belong until more information is available

TABLE 5
CLASS II HLA-RESTRICTED ANTIGENS

| | | | Tissue distribution | | |
|---|---|---|---|---|---|
| | HLA | | | Normal | |
| Gene | allele | Peptide epitope | Tumors | tissues | Reference |

Epitopes from normal protein antigens

| | | | | | |
|---|---|---|---|---|---|
| Amexin II | DRB*0401 | DVPKWISIM-TERSVPH | Melanoma | Not done | Li et al. |
| | | | | | 1998 |
| Gp100 | DRB1*0401 | WNRQLYPE- | Melanoma | Melanocytes | Li et al. |
| | | WTEAQRLD | | | 1998 |
| MAGE-1, -2, | DRB*1301 | LLKYRARIP- | Melanoma, lung and | Testis, | Chaux et |
| -3, -6 | DRB*1302 | VTKAE | breast carcinomas, | placenta | al. 1999a |
| | | | H/N SCC | | |
| MAGE-3 | DR*11-1 | TSYVKVLHHM-VKISG | Melanoma, lung and | Testis, | Manici et |
| | | | breast carcinomas, | placenta | al. 1999 |
| | | | H/N SCC | | |
| MAGE-3 | DRB*1301 | AELVHFLLLK- | Melanoma, lung and | Testis, | Chaux et |
| | DRB*1302 | YRAR | breast carcinomas, | placenta | al. 1999b |
| | | | H/N SCC | | |
| MART-1/ | DRB1*0401 | RNGYRALMDKS- | Melanoma | Melanocytes | Zarour et |
| Melan-A | | LHVGTQCALTRR | | | al. 2000 |
| MUCI | DR3 | PGSTAPPAHGVT | Breast and ovarian | None^{a} | Hitbold et |
| | | | cancers; multiple | | al. 1998 |
| | | | myeloma, B-cell | | |
| | | | lymphoma | | |
| NY-ESO-1 | DRB4*0101 | VLLKEFTVSG | Melanoma, B- | Testis | Zeng et al. |
| | | | lymphoma, | | 2000 |
| | | | hepatoma (Chen et | | |
| | | | al., 1997)^{b},sarcoma, | | |
| | | | H/N tumors, - | | |
| | | | bladder, lunch, | | |
| | | | prostate, ovarian, | | |
| | | | thyroid and breast | | |
| | | | carcinomas | | |
| NY-ESO-1 | DRB4*0101- | PLPVPGVLLK- | B-Lymphoma, | Testis | Jäger et |
| | 0103 | EFTVSNGI- | melanoma, sarcoma, | | al, 2000 |
| | | VLLKEFTVSG- | H/N tumors, | | |
| | | NILTIRLT | hepatoma (Chen et | | |
| | | AADHRQLQL- | al., 1997) - bladder, | | |
| | | SISSCLQQL | lung, prostate, | | |
| | | | ovarian, thyroid and | | |
| | | | breast carcinomas | | |
| PSA | DR4 | ILLGRMSLFM- | Melanoma | Melanocytes | Corman et |
| | | PEDTG | | | al. 1998 |
| | | SLFHPEDTGQVFQ | | | |
| | | QVFQVSHSFPHPLYD | | | |
| | | NDLMLLRLSEPAELT | | | |
| | | KKLQCVQLHVISM | | | |
| | | GVLQGITSMGSEPCA | | | |
| Tyrosinase | DRB1*0401 | QNILLSNAPLGPQFP | Melanoma | Melanocytes | Topalian |
| | | DYSYLQDSDPD- | | | et al. 1994 |
| | | SFQD | | | Topalian |
| | | SYLQDSDPDSFQD | | | et al. 1996 |
| Tyrosinase | DRB1*1501 | RHRPLQEVYP- | Melanoma | Melanocytes | Kobayashi |
| | | EANAPIGHNRE | | | et al. |
| | | | | | 1998a |
| Tyrosinase | DRB1*0405 | EIWRDIDFAHE | Melahoma | Melanocytes | Kobayashi |
| | | | | | et al. |
| | | | | | 1998b |

Epitopes from mutated protein antigens

| | | | | | |
|---|---|---|---|---|---|
| HPV-E7 | DR*0401 | LFMDTLSFVCPLC | Cervical carcinomas | None | Höhn et |
| | DR*0407 | LFMDSLNFVCPWC | | | al. 1999 |
| CDC27/m | DRB1*0401 | FSWAMDLDPKGA | Melanoma | None | Wang et |
| | | | | | al. |
| | | | | | 1999a |
| TPI/m | DRB1*0101 | GELIGILNAAKVPAD | Melanoma | None | Pieper |
| | | | | | et al. |
| | | | | | 1999 |

^{a}All epithelial tissues express highly glycosilated mucins whereas tumor cells often show hypoglycosilated mucins with a normal protein sequence.
^{b}Tissue distribution among tumors as described in the given references when different from the paper first reporting the sequence of the epitope.

V. Ex Vivo Culture of Dendritic Cells

A procedure for ex vivo expansion of hematopoietic stem and progenitor cells is described in U.S. Pat. No. 5,199,942, incorporated herein by reference. Other suitable methods are known in the art. Briefly, ex vivo culture and expansion comprises: (1) collecting CD34+ hematopoietic stem and progenitor cells from a patient from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used.

Stem or progenitor cells having the CD34 marker constitute only about 1% to 3% of the mononuclear cells in the bone marrow. The amount of CD34+ stem or progenitor cells in the peripheral blood is approximately 10- to 100-fold less than in bone marrow. Cytokines such as flt3-L may be used to increase or mobilize the numbers of dendritic cells in vivo. Increasing the quantity of an individual's dendritic cells may facilitate antigen presentation to T cells for antigen(s) that already exists within the patient, such as a disease antigen, such as a tumor antigen, or a bacterial or viral antigen. Alternatively, cytokines may be administered prior to, concurrently with or subsequent to administration of an antigen to an individual for immunization purposes.

Peripheral blood cells are collected using apheresis procedures known in the art. (See, for example, Bishop et al., 1994). Briefly, peripheral blood progenitor cells (PBPC) and peripheral blood stem cells (PBSC) are collected using conventional devices, for example, a Haemonetics Model V50 apheresis device (Haemonetics, Braintree, Mass.). Four-hour collections are performed typically no more than five times weekly until approximately 6.5×10⁸ mononuclear cells (MNC)/kg are collected. The cells are suspended in standard media and then centrifuged to remove red blood cells and neutrophils. Cells located at the interface between the two phases (the buffy coat) are withdrawn and resuspended in HBSS. The suspended cells are predominantly mononuclear and a substantial portion of the cell mixture are early stem cells.

A variety of cell selection techniques are known for identifying and separating CD34+ hematopoietic stem or progenitor cells from a population of cells. For example, monoclonal antibodies (or other specific cell binding proteins) can be used to bind to a marker protein or surface antigen protein found on stem or progenitor cells. Several such markers or cell surface antigens for hematopoietic stem cells (i.e., flt-3, CD34, My-10, and Thy-1) are known in the art, as are specific binding proteins therefore (see for example, U.S. Ser. No. 08/539,142, filed Oct. 4, 1995).

In one method, antibodies or binding proteins are fixed to a surface, for example, glass beads or flask, magnetic beads, or a suitable chromatography resin, and contacted with the population of cells. The stem cells are then bound to the bead matrix. Alternatively, the binding proteins can be incubated with the cell mixture and the resulting combination contacted with a surface having an affinity for the antibody-cell complex. Undesired cells and cell matter are removed providing a relatively pure population of stem cells. The specific cell binding proteins can also be labeled with a fluorescent label, e.g., chromophore or fluorophore, and the labeled cells separated by sorting. Preferably, isolation is accomplished by an immunoaffinity column.

Immunoaffinity columns can take any form, but usually comprise a packed bed reactor. The packed bed in these bioreactors is preferably made of a porous material having a substantially uniform coating of a substrate. The porous material, which provides a high surface area-to-volume ratio, allows for the cell mixture to flow over a large contact area while not impeding the flow of cells out of the bed. The substrate should, either by its own properties, or by the addition of a chemical moiety, display high-affinity for a moiety found on the cell-binding protein. Typical substrates include avidin and streptavidin, while other conventional substrates can be used.

In one useful method, monoclonal antibodies that recognize a cell surface antigen on the cells to be separated are typically further modified to present a biotin moiety. The affinity of biotin for avidin thereby removably secures the monoclonal antibody to the surface of a packed bed (see Berenson et al., 1986). The packed bed is washed to remove unbound material, and target cells are released using conventional methods. Immunoaffinity columns of the type described above that utilize biotinylated anti-CD34 monoclonal antibodies secured to an avidin-coated packed bed are described for example, in WO 93/08268.

An alternative means of selecting the quiescent stem cells is to induce cell death in the dividing, more lineage-committed, cell types using an antimetabolite such as 5-fluorouracil (5-FU) or an alkylating agent such as 4-hydroxycyclophosphamide (4-HC). The non-quiescent cells are stimulated to proliferate and differentiate by the addition of growth factors that have little or no effect on the stem cells, causing the non-stem cells to proliferate and differentiate and making them more vulnerable to the cytotoxic effects of 5-FU or 4-HC. (See Berardi et al., 1995), which is incorporated herein by reference.)

Isolated stem cells can be frozen in a controlled rate freezer (e.g., Cryo-Med, Mt. Clemens, Mich.), then stored in the vapor phase of liquid nitrogen using dimethylsulfoxide as a cryoprotectant. A variety of growth and culture media can be used for the growth and culture of dendritic cells (fresh or frozen), including serum-depleted or serum-based media. Useful growth media include RPMI, TC 199, Iscoves modified Dulbecco's medium (Iscove et al., 1978), DMEM, Fischer's, alpha medium, NCTC, F-10, Leibovitz's L-15, MEM and McCoy's.

Particular nutrients present in the media include serum albumin, transferrin, lipids, cholesterol, a reducing agent such as 2-mercaptoethanol or monothioglycerol, pyruvate, butyrate, and a glucocorticoid such as hydrocortisone 2-hemisuccinate. More particularly, the standard media includes an energy source, vitamins or other cell-supporting organic compounds, a buffer such as HEPES, or Tris, that acts to stabilize the pH of the media, and various inorganic salts. A variety of serum-free cellular growth media is described in WO 95/00632, which is incorporated herein by reference.

The collected CD34.+ cells are cultured with suitable cytokines, for example, as described herein, and in U.S. Ser. No. 08/539,142. CD34+ cells then are allowed to differentiate and commit to cells of the dendritic lineage. These cells are then further purified by flow cytometry or similar means, using markers characteristic of dendritic cells, such as CD1 a, HLA DR, CD80 and/or CD86. The cultured dendritic cells are exposed to an antigen, for example, a tumor antigen or an antigen derived from a pathogenic or opportunistic organism, allowed to process the antigen, and then cultured with an amount of a CD40 binding protein to activate the dendritic cell. Alternatively, the dendritic cells are transfected with a gene encoding an antigen, and then cultured with an amount of a CD40 binding protein to activate the antigen-presenting dendritic cells.

VI. Identifying T Cell Epitopes

To identify peptides that are recognized by either CD4+ or CD8+T cells, a series of overlapping peptides (for example, about 9-13 amino acids) will be made based on the predicted amino acid sequence from a gene of interest. T cells recognize a peptide bound to the MHC class I or II molecules. The synthetic peptides are then tested for their ability to stimulate cytokine secretion from T cells when pulsed onto MHC matched antigen presenting cells (APC) such as EBV transformed B cells or dendritic cells. Once positive peptides are identified, a series of truncations are made at the N- and C-terminus of a peptide such that a minimal length of a T-cell peptide is defined.

VII. Kits

Any of the compositions described herein may be comprised in a kit. In a non-limiting example, a composition comprising a dendritic cell having a cell penetrating peptide associated with an antigen, such as a tumor antigen, and/or additional agent, may be comprised in a kit. In a specific embodiment, the composition is a vaccine. In another specific embodiment the cell penetrating peptide and/or the tumor antigen are housed in a kit, and the dendritic cell is provided elsewhere, such as derived from the patient being treated with the kit component(s). The kits will thus comprise, in suitable container means, a dendritic cell, a cell penetrating peptide, and/or an antigen. In a specific embodiment, the kit is tailored to a disease, wherein the antigen is specific for the disease. In the embodiment wherein the kit is for cancer treatment, the kit contains a tumor antigen associated with a particular cancer.

The kits may comprise suitably aliquoted dendritic cells and/or related components of the invention compositions of the present invention, whether labeled or unlabeled, as may be used to prepare a standard curve for a detection assay. Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the kit components in their containers in close confinement for commercial sale. Such containers may include injection or blow-molded plastic containers into which the desired vials are retained.

The kits will generally contain, in suitable container means, a pharmaceutically acceptable formulation of the dendritic cell, cell penetrating peptide, and/or antigen. The kit may have a single container means, and/or it may have distinct container means for each compound. When the components of the kit are provided in one and/or more liquid solutions, the liquid solution is an aqueous solution, with a sterile aqueous solution being particularly preferred. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile, pharmaceutically acceptable buffer and/or other diluent.

Irrespective of the number and/or type of containers, the kits of the invention may also comprise, and/or be packaged with, an instrument for assisting with the injection/administration and/or placement of the ultimate composition(s) within the body of an animal. Such an instrument may be a syringe, pipette, forceps, and/or any such medically approved delivery vehicle.

VIII. Immunotherapeutic Agents

The disclosures presented herein have significant relevance to immunotherapy of human diseases and disorders, including cancer. In using the immunotherapeutic compositions of the present invention in treatment methods for cancer, other standard treatments also may be employed, such as radiotherapy or chemotherapy. However, in specific embodiments additional immunotherapy methods may also be used. Some immunotherapies of cancer are described in the following sections and may be used or produced with the methods and compositions of the present invention.

An immunotherapeutic agent generally relies on the use of immune effector cells and molecules to target and destroy cancer cells. In one aspect of immunotherapy, the tumor cell must bear some marker that is amenable to targeting, i.e., is not present on the majority of other cells. Many tumor markers exist and any of these may be suitable for targeting in the context of the present invention.

A. Immune Stimulators

A specific aspect of immunotherapy is to use an immune stimulating molecule as an agent, or more preferably in conjunction with another agent, such as, for example, a cytokine such as, for example, IL-2, IL-4, IL-12, GM-CSF, tumor necrosis factor; interferons alpha, beta, and gamma; F42K and other cytokine analogs; a chemokine such as, for example, MIP-1, MIP-1beta, MCP-1, RANTES, IL-8; or a growth factor such as, for example, FLT3 ligand.

One particular cytokine contemplated for use in the present invention is tumor necrosis factor. Tumor necrosis factor (TNF; Cachectin) is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

Another cytokine specifically contemplated is interferon alpha. Interferon alpha has been used in treatment of hairy cell leukemia, Kaposi's sarcoma, melanoma, carcinoid, renal cell cancer, ovary cancer, bladder cancer, non-Hodgkin's lymphomas, mycosis fungoides, multiple myeloma, and chronic granulocytic leukemia.

B. Active Immunotherapy

In active immunotherapy, an antigenic peptide, polypeptide or protein, or an autologous or allogenic tumor cell composition or “vaccine” is administered, generally with a distinct bacterial adjuvant (Ravindranath & Morton, 1991; Morton & Ravindranath, 1996; Morton et al., 1992; Mitchell et al., 1990; Mitchell et al., 1993). In melanoma immunotherapy, those patients who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

C. Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated anigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma.

IX. Enhancement of an Immune Response

The present invention includes a method of enhancing the immune response in a subject comprising the steps of contacting one or more lymphocytes with an antigenic composition, wherein the antigen is presented by an immune system cell, such as the dendritic cells of the present invention, and in a specific embodiment comprises as part of its sequence a sequence in accordance with SEQ ID NO:1, or an immunologically functional equivalent thereof. As used herein, an “antigenic composition” may comprises an antigen (e.g., a peptide). In certain embodiments, the antigenic composition is conjugated to or comprises an HLA anchor motif amino acids.

In other embodiments, the compositions of the present invention are in a mixture that comprises an additional immunostimulatory agent or nucleic acids encoding such an agent. Immunostimulatory agents include but are not limited to an additional antigen, an immunomodulator, an antigen presenting cell or an adjuvant. In other embodiments, one or more of the additional agent(s) is covalently bonded to the antigen or an agent, in any combination.

The enhanced immune response may be an active or a passive immune response. Alternatively, the response may be part of an adoptive immunotherapy approach in which immune system cells, such as dendritic cells, B cells or macrophages, are obtained from an animal (e.g., a patient), then pulsed with composition comprising an antigenic composition. In this embodiment, the antigenic composition may comprise an additional immunostimulatory agent or a nucleic acid encoding such an agent. In a preferred embodiment, the animal (e.g., a patient) has or is suspected of having a cancer. In other embodiments the method of enhancing the immune response is practiced in conjunction with a cancer therapy, such as for example, a cancer vaccine therapy.

In one embodiment, an antigen presenting cell is utilized in the present invention. In general, the term “antigen presenting cell” can be any cell that accomplishes the goal of the invention by aiding the enhancement of an immune response (i.e., from the T-cell or −B-cell arms of the immune system) against an antigen (e.g., a tumor antigen) or antigenic composition of the present invention. Such cells can be defined by those of skill in the art, using methods disclosed herein and in the art. As is understood by one of ordinary skill in the art (see for example Kuby, 1993, incorporated herein by reference), and used herein certain embodiments, a cell that displays or presents an antigen normally or preferentially with a class II major histocompatability molecule or complex to an immune cell is an “antigen presenting cell.” In certain aspects, a cell (e.g., an APC cell) may be fused with another cell, such as a recombinant cell or a tumor cell that expresses the desired antigen. Methods for preparing a fusion of two or more cells is well known in the art, such as for example, the methods disclosed in Goding, pp. 65-66, 71-74 1986; Campbell, pp. 75-83, 1984; Kohler and Milstein, 1975; Kohler and Milstein, 1976, Gefter et al., 1977, each incorporated herein by reference. In some cases, the immune cell to which an antigen presenting cell displays or presents an antigen to is a CD4⁺T_{H} cell. Additional molecules expressed on the APC or other immune cells may aid or improve the enhancement of an immune response. Secreted or soluble molecules, such as for example, cytokines and adjuvants, may also aid or enhance the immune response against an antigen. Such molecules are well known to one of skill in the art, and various examples are described herein.

X. Combination Treatments

In order to increase the effectiveness of the therapeutic compositions of the present invention, it may be desirable to combine these compositions with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

A. Gene Therapy

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that the compositions could be used similarly in conjunction with chemotherapeutic, radiotherapeutic, or immunotherapeutic intervention, in addition to other pro-apoptotic or cell cycle regulating agents.

Alternatively, the gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, gene therapy is “A” and the secondary agent, such as radio- or chemotherapy, is “B”: A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

B. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

C. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

D. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with the methods and/or compositions of the present invention. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs'surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

XI. Biological Functional Equivalents

The present invention comprises methods and compositions concerning peptides. As modifications and/or changes may be made in the structure of the peptides of the present invention, it is within the scope of the present invention to include biologically functional equivalent molecules having similar or improved characteristics. A skilled artisan recognizes that a biologically functional equivalent of a cell penetrating peptide is one in which it is still capable of facilitating entry of another peptide, such as the antigenic peptide, into a cell. A skilled artisan also recognizes that a biologically functional equivalent of a self-antigen peptide is one in which it is capable of enhancing an immune response when presented by the immune effector cell. In a specific embodiment, this includes providing protection against a subsequent challenge of the disease or pathogen.

A. Modified Peptides

Certain amino acids may be substituted for other amino acids in a peptide without appreciable loss of interactive binding capacity with structures such as, for example, binding sites on substrate molecules, receptors, and such like. So-called “conservative” changes do not disrupt the biological activity of the protein, as the structural change is not one that impinges on the peptide's ability to carry out its designed function. It is thus contemplated by the inventors that various changes may be made in the sequence of peptides disclosed herein, while still fulfilling the goals of the present invention.

In terms of functional equivalents, it is well understood by the skilled artisan that, inherent in the definition of a “biologically functional equivalent” protein and/or polynucleotide, is the concept that there is a limit to the number of changes that may be made within a defined portion of the molecule while retaining a molecule with an acceptable level of equivalent biological activity.

Amino acid substitutions are generally based on the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and/or histidine are all positively charged residues; that alanine, glycine and/or serine are all a similar size; and/or that phenylalanine, tryptophan and/or tyrosine all have a generally similar shape. Therefore, based upon these considerations, arginine, lysine and/or histidine; alanine, glycine and/or serine; and/or phenylalanine, tryptophan and/or tyrosine; are defined herein as biologically functional equivalents.

To effect more quantitative changes, the hydropathic index of amino acids may be considered. Each amino acid has been assigned a hydropathic index on the basis of their hydrophobicity and/or charge characteristics, these are: isoleucine (+4.5); valine (|4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and/or arginine (−4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index and/or score and/or still retain a similar biological activity. In making changes based upon the hydropathic index, the substitution of amino acids whose hydropathic indices are within±2 is preferred, those which are within±1 are particularly preferred, and/or those within±0.5 are even more particularly preferred.

It also is understood in the art that the substitution of like amino acids can be made effectively on the basis of hydrophilicity, particularly where the biological functional equivalent protein and/or peptide thereby created is intended for use in immunological embodiments, as in certain embodiments of the present invention. U.S. Pat. No. 4,554,101, incorporated herein by reference, states that the greatest local average hydrophilicity of a protein, as governed by the hydrophilicity of its adjacent amino acids, correlates with its immunogenicity and/or antigenicity, i.e., with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within±2 is preferred, those which are within±1 are particularly preferred, and/or those within±0.5 are even more particularly preferred.

B. Altered Amino Acids

The present invention, in many aspects, relies on peptides. These peptides may include the twenty “natural” amino acids, and post-translational modifications thereof. However, in vitro peptide synthesis permits the use of modified and/or unusual amino acids. A table of exemplary, but not limiting, modified and/or unusual amino acids is provided herein below.

TABLE 6
Modified and/or Unusual Amino Acids

| Abbr. | Amino Acid | Abbr. | Amino Acid |
|---|---|---|---|
| Aad | 2-Aminoadipic acid | EtAsn | N-Ethylasparagine |
| BAad | 3-Aminoadipic acid | Hyl | Hydroxylysine |
| BAla | beta-alanine, beta-Amino-propionic | AHyl | allo-Hydroxylysine |
| | acid | | |
| Abu | 2-Aminobutyric acid | 3Hyp | 3-Hydroxyproline |
| 4Abu | 4-Aminobutyric acid, piperidinic | 4Hyp | 4-Hydroxyproline |
| | acid | | |
| Acp | 6-Aminocaproic acid | Ide | Isodesmosine |
| Ahe | 2-Aminoheptanoic acid | Aile | allo-Isoleucine |
| Aib | 2-Aminoisobutyric acid | MeGly | N-Methylglycine, |
| | | | sarcosine |
| BAib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

C. Mimetics

In addition to the biological functional equivalents discussed above, the present inventors also contemplate that structurally similar compounds may be formulated to mimic the key portions of peptide or polypeptides of the present invention. Such compounds, which may be termed peptidomimetics, may be used in the same manner as the peptides of the invention and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Methods for generating specific structures have been disclosed in the art. For example, alpha-helix mimetics are disclosed in U.S. Pat. Nos. 5,446,128; 5,710,245; 5,840,833; and 5,859,184. Theses structures render the peptide or protein more thermally stable, also increase resistance to proteolytic degradation. Six, seven, eleven, twelve, thirteen and fourteen membered ring structures are disclosed.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Experimental Protocols

Cell Lines. B16 is a pigmented mouse melanoma cell line of C57BL/6 origin. MC-38 is an adenocarcinoma cell line. 293 Kb is a transfected 293 cell line expressing the murine MHC class I Kb molecule (a gift of Dr. James Yang at NCI). Cell lines were maintained at 37° C. and 5% CO2 in RPMI 1640 supplemented with 10% heat-inactivated fetal bovine serum, 2 mM glutamine, 100 U/ml penicillin, 100 μg/ml streptomycin (Biofluids, Rockville, Md.), 2.5 mg/ml of Fungizone (GIBCO BRL, Gaithersburg, Md.).

Peptides. The TRP2 peptide used in this study is a 9 amino acid sequence (SVYDFFVWL), derived from the TRP2 protein. Control H2-Kb-restricted peptide was beta-galactosidase (βgal) (DAPIYTNV). The cell-penetrating peptide used in this study is a 12-mer CPP (AAVLLPVLLAAP), designated CPP1. The CPP1-TRP2 (AAVLLPVLLAAPSVYDFFVWL) and CPP1-β-gal (AAVLLPVLLAAPDAPIYTNV) peptides were synthesized and purified by HPLC. All peptides were dissolved in DMSO, and diluted in PBS for final concentrations.

Dendritic Cell Preparation and Analysis. Mouse immature and mature DCs were derived from C57BL/6 bone marrow as previously described (Labeur et al., 1999; Specht et al., 1997). Phenotype and mixed leukocyte reaction activity of DCs were analyzed according to the protocol described (Specht et al., 1997). Briefly, immature DCs were generated in the presence of murine GM-CSF without transfer steps. The loosely adherent immature DCs were harvested on day 5 and used for experiments. Mature DCs were generated in the presence of recombinant GM-CSF and 1000 U/ml of IL-4 (Peprotech, Rocky Hill, N.J.). The loosely adherent cells on day 6 were transferred to 10-cm petri dishes. Nonadherent cells were harvested and pulsed for 2 h at 37° C. with peptides in Opti-MEM media (GIBCO BRL, Gaithersburg, Md.), washed three times with PBS and used for mouse injections (3×10⁵ cells intravenously per mouse). Immature and mature DCs were stained with antibodies against MHC class I and MHC class II molecules followed by FITC-conjugated goat anti-mouse IgG2a, anti-B7 antibody followed by PE-conjugated goat antihuman IgG, CD40 followed by PE-conjugated goat anti-rat IgG, or anti-CD11c ((PharMingen, San Diego, Calif.). Cells were then analyzed on a flow cytometer (FACScan™, Becton-Dickinson, San Jose, Calif.). Typically, immature DCs we generated exhibited a relatively low expression of MHC class II and costimulatory molecules, while mature DCs expressed a high level of class II and costimulatory molecules, and displayed potent capacities for stimulating allogeneic T cells in vitro compared with immature DCs and splenocytes.

Mice and Tumor Treatment. Six to eight week-old female C57BL/6 (B6), CD4 knock out (KO) and CD8 KO mice were purchased from The National Cancer Institute and Taconic, and maintained in a pathogen-free mice facility at Baylor College of Medicine. For the 3-day tumor model, all mice were injected intravenously through the tail vein with 3×10⁵ B16 melanoma cells. After three days, mice were injected intravenously with 3×10⁵ peptide-loaded DCs. Fourteen days later, lungs were removed and metastases enumerated in a blinded fashion. For the prevention model, tumor challenge was performed 14 days after immunization with DC/peptides. Two weeks later, mice were sacrificed, all lobes of both lungs were dissected, and metastases were counted. For antibody depletion, 200 μg of anti-CD4 (GK1.5), anti-CD8 (2.43) or control antibodies in 500 μl were i.p. injected for each mouse on the day before tumor challenge, and followed by three injections on day 1, 3 and 10 post tumor injection. Depletion of CD4+ or CD8+T cells was determined by FACS analysis.

T Cell Activity against DCs Loaded with Peptides. Mature DCs were pulsed with 10 μM of each peptide for 2 h at 37° C. Following three washes with T cell assay medium (RPMI 1640 containing 5% human AB serum, glutamine and 120 IU of IL-2, 1×10⁵ DCs/peptide were cocultured with TRP2-specific CTLs (1×10⁵) for 12 h at 37° C. and 5% CO₂. The remaining peptide-loaded DCs were span down and resuspended in DC growth medium. At different time points (4, 18 and 24 h after peptide pulsing), these peptide-loaded DCs were harvested, washed once and the same number of cells were cocultured with CTLs. All cell culture supernatants were harvested after 12 h incubation and stored at −20° C. until use.

Cytokine Release Assay. Two weeks after immunization, splenocytes were harvested from mice that had not been challenged with tumor. The splenocytes were restimulated with the TRP2 peptide in vitro (1 μg/ml) on the first day of culture. The cell culture was maintained for 6 days and IL-2 was added on the third day of culture. T cells were tested against 293 Kb pulsed with TRP2 or β-gal peptide (1 μg/ml) or against B16 tumor. Supernatants were harvested after overnight incubation and measured for murine IFN-gamma. or GM-CSF release by ELISA (Endogen, Woburg, Mass.) according to the manufacturer's instructions.

### Example 2

### DCs Pulsed with a TRP2 Self-Peptide Fail To Elicit Potent Antitumor Immunity

In previous studies, vaccination of mice with TRP2 peptide in incomplete Freund's adjuvant (IFA), DNA, adenovirus or vaccinia virus encoding murine TRP2 failed to produce protective or therapeutic immunity against B16 tumor (Schreurs et al., 2000; Zeh et al., 1999; Overwijk et al., 1999; Bowne et al., 1999), although cytotoxic T lymphocytes (CTLs) specific for the TRP2 peptide were readily generated in vitro from the spleens of mice immunized with the TRP2 peptide plus IFA (Zeh et al., 1999).

Mature DCs pulsed with peptides derived from model antigens were tested for enhancement of anti-tumor immunity against B16 tumor cells. C57BL/6 (B6) mice were immunized by a single intravenous injection of 3×10⁵ DCs pulsed with the TRP2 self-peptide. Two weeks later, the immunized mice were challenged with a lethal dose (3×10⁵) of B16 tumor cells. Lungs were harvested and lung metastases counted after 2 weeks of B16 tumor challenge. Little improvement in protective immunity was observed in mice immunized with the TRP-2 peptide-pulsed DCs compared with mice immunized with DCs alone or DC pulsed with a control peptide.

### Example 3

### CPP1 and its Use to Deliver TRP2 Peptide into DC's for Prolonging Antigen Presentation

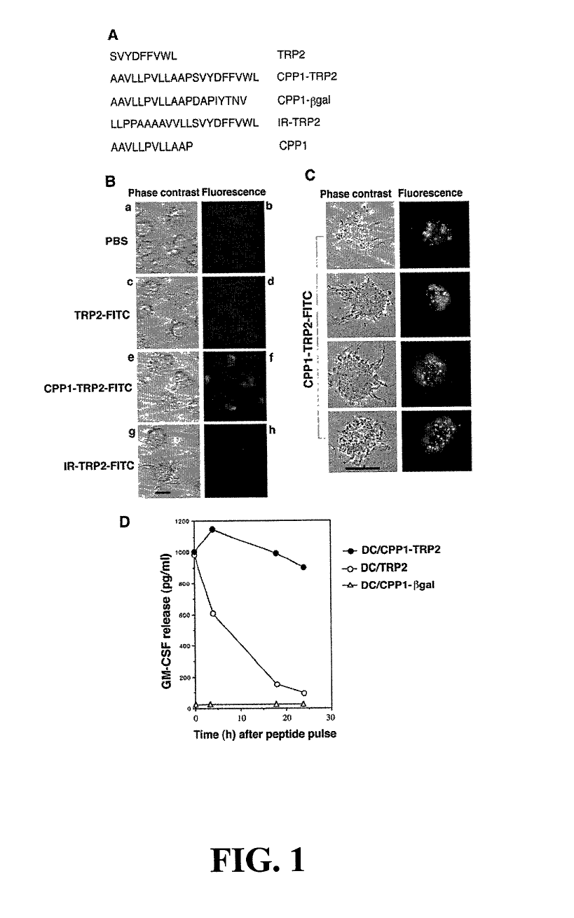

To overcome the problem of peptide disassociation from DCs, the TRP2 peptide was delivered into mature DCs intracellularly. MTS (AAVLLPVLLAAP) fused proteins have been shown to rapidly and efficiently enter intact cells (Rojas et al., 1998). The MTS sequence was designated as the cell-penetrating peptide 1 (CPP1), and in specific embodiments, when covalently linked to a self-peptide, it has dual functions: 1) facilitating delivery of the self peptide into mature DCs, and 2) protecting the self peptide from protease degradation during in vitro pulsing. Once inside the mature DCs, the internalized CPP1-linked self-peptides in specific embodiments are processed and presented by newly synthesized MHC class I molecules on the DC surface for T cell recognition. Therefore, in preferred embodiments this approach allows mature DCs to present a self-peptide for longer times and at a high density in vivo, leading to the generation of strong antitumor immunity. Several peptides: CPP1-TRP2, CPP1-βgal, TRP2, and irrelevant peptide (IR)-TRP2 (FIG. 1A) were chemically synthesized.

These peptides were tested for the ability to enter mature DCs, and they were labeled the N-terminus with fluorescein and incubated them with mature DC. The CPP1-TRP2 peptide was efficiently internalized, but other peptides failed to enter mature DCs efficiently (FIG. 1B). Furthermore, it was found that the CPP1-TRP2 peptide had translocated and accumulated in the cytoplasm as well as the cell nucleus. The internalized peptides were localized in vesicles such as endosomes based on distribution in the cell, rather than free in the cytoplasm (FIG. 1C).

To test whether CPP1-TRP2 would prolong the presentation of MHC/peptide complexes to T cells, mature DCs were pulsed with TRP2, CPP1-TRP2 and CPP1-βgal (control peptide) for 2 h at 37° C. After washing, some peptide-loaded DCs were immediately mixed with TRP2-specific T cells (0 h), and incubated for additional 12 h. The remaining peptide-loaded DCs were mixed with T cells later at different time points, i.e. 4, 18 and 24 h. All cell cultures were incubated for 12 h before harvesting for ELISA assay. T cell activities for DC/TRP2 and DC/CPP1-TRP2 were almost the same at 0 h after peptide pulsing. There was no T cell activity against DC/CPP1-βgal (FIG. 1D), suggesting that T cells were specific for the TRP2 peptide. Four hours after peptide pulsing, however, DC/TRP2 had lost 40% of its ability to stimulate T cells for GM-CSF release. At 18 and 24 h after peptide pulsing, DC/TRP2 had lost >90% of their ability to activate T cells. By contrast, at 4 h following peptide pulsing, DC/CPP1-TRP2 had slightly increased its capacity to stimulate the secretion of GM-CSF release from T cells. More importantly, DC/CPP1-TRP2 retained almost full capacity to present MHC/TRP2 complexes on the cell surface to T cells for at least 24 h (FIG. 1D). Thus, the internalization of tumor antigen peptides into DCs enhances the presentation of MHC/peptide complexes to T cells.

### Example 4

### Immunization with DCS Loaded with CPP1-TRP2 Protects Mice From B16 Tumor Challenge

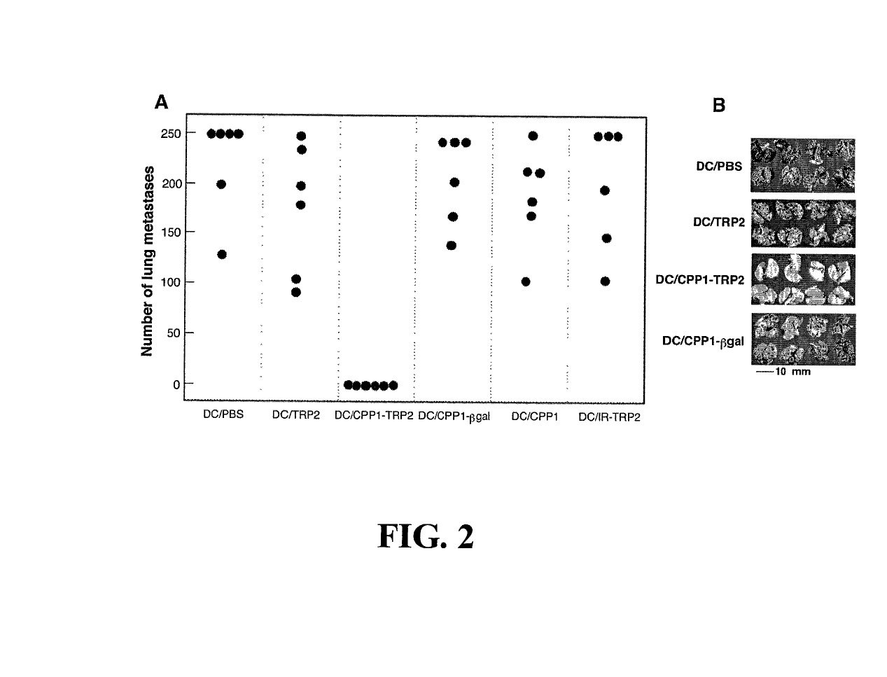

Mature DCs loaded with CPP1-TRP2 peptide were tested for generation of potent protective immunity against B16 tumor. Mice were injected intravenously with a single dose of DCs loaded with various peptides. Two weeks later, the immunized mice were challenged with 3×10⁵ B16 tumor cells. After 2 weeks of tumor challenge, the lungs of the immunized mice were harvested and lung metastases counted. None of the mice immunized with DC/CPP1-TRP2 had tumor metastases, while those mice immunized with DC alone or DC/control peptides developed more than 100 lung metastases (FIG. 2A). Notably, immunization with DC/TRP2 failed to protect mice from B16 tumor challenge. Similar results were obtained in three separate experiments. FIG. 2B is a representative of gross pathology of lungs for groups of mice that received each treatment. Thus, the prolonged antigen presentation of DCs through intracellular delivery of peptide considerably enhanced antitumor responses in B16 tumor model.

### Example 5

### Requirement of Mature DCS Loaded with CPP1-TRP2 for Potent Immunity

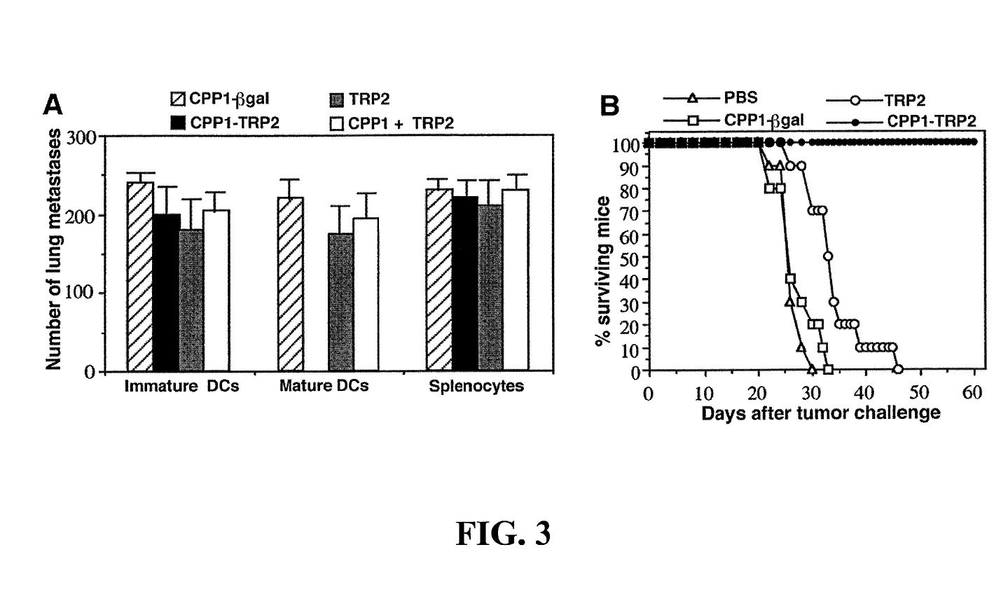

Immature DCs and splenocytes loaded with the CPP1-TRP2 peptide were tested for the ability to induce protective antitumor immunity. Immature DCs, mature DCs and splenocytes were prepared and loaded with TRP2, CPP1-TRP2, TRP2 plus CPP1, or CPP1-βgal, and used them to immunize mice. Neither peptide-loaded splenocytes nor peptide-loaded immature DCs were effective in inhibiting B16 tumor growth (FIG. 3A). In contrast, mature DCs loaded with CPP1-TRP2 effectively eliminated B16 tumor cells, while DCs loaded with TRP2 or other control peptides did not produce protective antitumor responses. These results indicate that both mature DCs and CPP1-TRP2 are required for the induction of protective antitumor immunity against B16 tumor cells. Peptide-pulsed immature DCs and splenocytes were not effective in generating potent immunity. These findings were consistent with other studies showing that mature DCs were better APC than immature DCs and splenocytes in eliciting T cell responses against tumor cells (Labeur et al., 1999; Winzler et al., 1997).

While a single injection of DCs/CPP1-TRP2 completely eliminated B16 tumor challenge, DCs/TRP2 vaccination failed to eliminate B16 tumor (FIGS. 2A and 2B). It was next tested whether multiple injections could improve T cell response and found that at least two injections of DCs/TRP2 were required to produce a weak protective immunity that eliminate B16 tumor in 2 of 5 mice. To further compare if the protective immunity elicited by DCs/CPP1-TRP2 1 or DCs/TRP2 could prolong mice survival after B16 tumor challenge, mice received two injections of DCs/PBS, DCs/CPP1-βgal, DCs/TRP2 and DCs/CPP1-TRP2. Two weeks after the last injection, the immunized mice were challenged with B16 tumor. Animal survival was then monitored. As shown in FIG. 3B, DCs/TRP2 vaccines showed some delay of animal survival compared with groups received injections of DCs/PBS or DCs/CPP1-βgal. In contrast, DCs/CPP1-TRP2 prolonged animal survival for at least 60 days of B16 tumor challenge. Thus, these results indicated that DCs/CPP1-TRP2 vaccination elicited potent protective immunity, and significantly prolonged the animal survival after tumor challenge. On the other hand, multiple vaccinations of DC/TRP2 were required to produce partial protective immunity and to slightly prolong the survival of mice after tumor challenge, which is consistent with results obtained by other groups (Bellone et al., 2000; Schreurs et al., 2000).

### Example 6

### Induction of CD8+ T Cells After Immunization

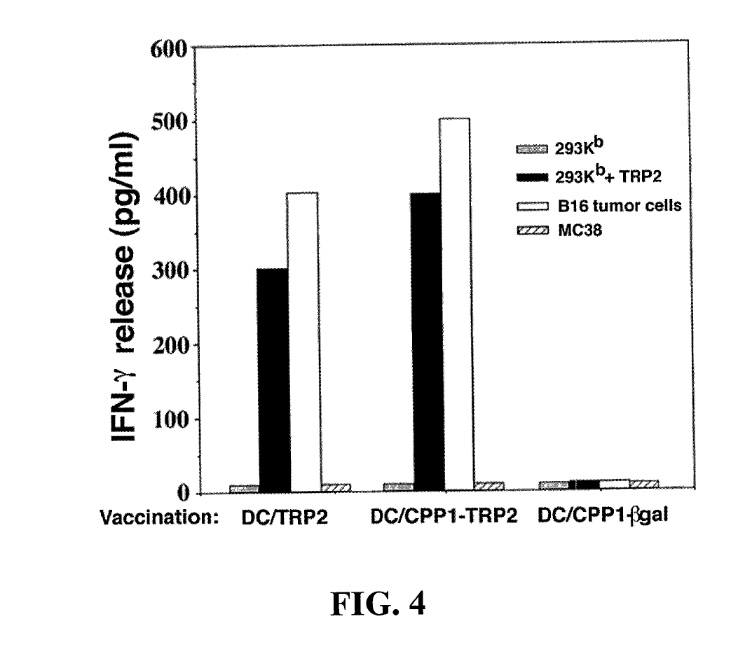

Mice immunized with DCs loaded with peptides were tested for the ability to generate TRP-2-specific CD8+ T cells and whether these in vitro activities correlated with antitumor activity in vivo. Splenocytes were harvested from mice immunized with DCs loaded with different peptides and stimulated with the TRP2 peptide in vitro. T cell activity was measured using 293 Kb pulsed with the TRP2 peptide as well as B16 tumor cells as targets. CD8+ T cells generated from mice immunized with either DC/TRP2 or DC/CPP1-TRP2 were capable of recognizing both peptide-pulsed targets and B16 tumor cells, but not MHC class I-matched MC38 tumor cells or 293 Kb cells (FIG. 4). These CD8+ T cells also lysed B16 tumor cells, but not MC38 tumor cells. By contrast, none of the mice vaccinated with DCs loaded with a control peptide (CPP1-βgal) had TRP2-specific CTLs.

These results suggest that TRP-2-specific CD8+ T cells could be elicited after vaccination with either DC/TRP2 or DC/CPP1-TRP2, but the T cell activity detected in vitro did not correlate with antitumor activity in vivo shown in FIG. 2. Previous studies showed that CD8+ T cell responses could be induced by peptide in IFA and naked DNA, but they failed to produce protective immunity in vivo (Bellone et al., 2000; Zeh et al., 1999). Furthermore, multiple immunization of DC/TRP2 (SVYDFFVWL 9-mer) or TRP2 (VYDFFVWL 8-mer) generated the comparable CTL activity in vitro, but the level of protective immunity in vivo was higher for the high-affinity Kb binding peptide (SVYDFFVWL 9-mer) (Schreurs et al., 2000), implying that prolonging presentation of peptide on the cell surface could improve immune response in vivo. Similar results were obtained in other animal tumor models as well as human clinical setting (Thurner et al., 1999; Rosenberg et al., 1998; Srivastava, 2000). Since TRP2 is a self-antigen, T cells with high affinity for MHC-peptide complexes may have been deleted due to central and peripheral tolerance. To induce effective antitumor immunity, it may be necessary to prolonging antigen presentation on DCs. It appeared that intracellular delivery of peptide into DCs allows not only naturally processing of synthetic peptides through endogenous pathways, but also prolonging antigen presentation for T cell activation. Combination of these effects may improve in vivo T cell responses against cancer, but did not reflect T cell activity detected in vitro through a mechanism that is not understood at the present time. There are several possible explanations for the phenomenon. T cell trafficking in vivo (tumor sites versus splenocytes or PBMCs) and T cell apoptosis during in vitro peptide stimulation in a specific embodiment attributes to the difference in antitumor activity between in vitro and in vivo (Rosenberg et al., 1998; Suhrbier et al., 1993). Finally, other effectors such as CD4+ T cells in some embodiments explain why DC/CPP1-TRP2 was effective in inducing potent antitumor activity.

### Example 7

### Both CD4+ and CD8+ T Cells are Required for a Protective Immunity

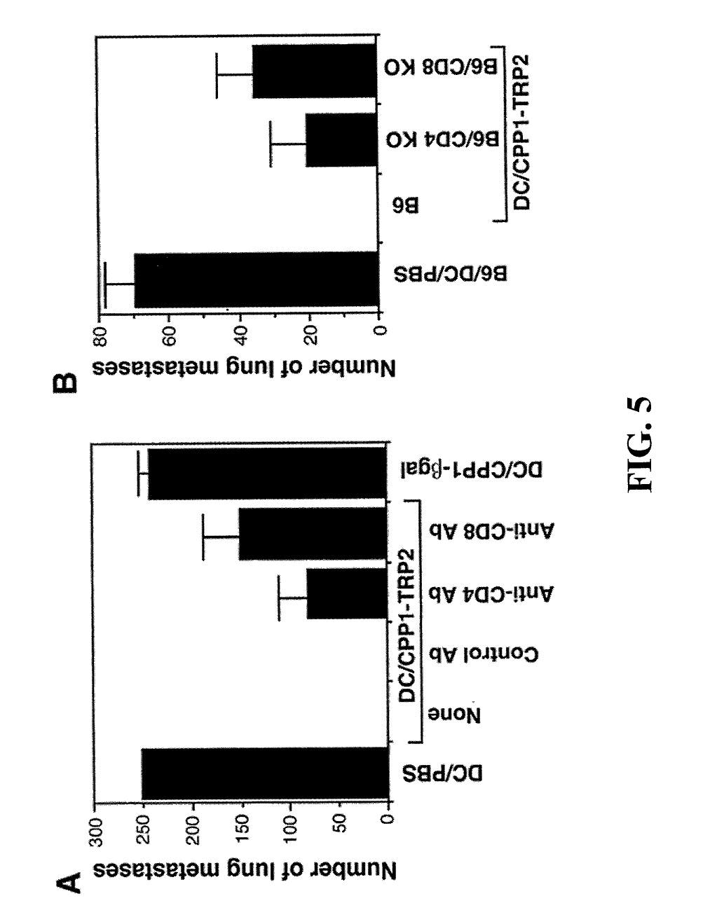

To test whether CD4+ T cell were required for the protective immunity in vivo, CD4+ or CD8+ T cells were depleted in mice previously immunized with DC/CPP1-TRP2 peptide by specific antibodies against CD4 or CD8 molecules and a control antibody. Antibodies were i.p injected on the day before B16 tumor challenge, and followed by three injections on day 1, 3 and 10 after tumor injection. Immunization with DC/CPP1-TRP2 completely protected mice from tumor challenge. Potent antitumor immunity was not compromised in mice treated with the control antibody. However, depletion of CD8+ T cells by anti-CD8 antibody strikingly diminished the ability to protect mice from tumor challenge (FIG. 5A). Depletion of CD4+ T cells also affected the ability of the immunized mice to reject tumor cells.

To further test the role of CD4+ T cells, wild-type as well as CD4 and CD8 knock-out (KO) B6 mice were immunized with CPP1-TRP2-loaded DCs. Genetically intact B6 mice immunized with DC/PBS and DC/CPP1-βgal failed to reject tumor challenge, while those vaccinated with DC/CPP 1 -TRP2 completely rejected B16 tumor cells (FIG. 5B). However, CD4 KO and CD8 KO mice received the DCs/CPP1-TRP2 vaccine failed to eliminate B16 tumor, resulting in increase in the number of lung metastases in both groups compared with wild type mice received the same vaccine. Taken together, these results suggest that both CD4+ and CD8+ T cells are required for antitumor responses.

One may hypothesize that the CPP1-TRP2 (21-mer) peptide may induce peptide-specific CD4+ T cells, while the TRP2 (9-mer) peptide fails to do so. However, CPP1-TRP2-specfic or TRP2-specific CD4+ T cell activity was not detected. CD4+ T cells have been demonstrated to be required for antitumor effect in vivo elicited by both TRP1 and TRP2 (Houghton et al., 2001), but their direct involvement or exact mechanisms are not clear at the present time. A recent study showed that both tumor-specific and non-specific (unrelated) T helper peptides could provide critical help for eliciting CTL immunity (Casares et al., 2001). The importance of CD4+ T cells in immune responses against cancer has been widely accepted (Wang, 2001; Greenberg, 1991; Pardoll and Tapolaian, 1998; Hung et al., 1998; Toes et al., 1999; Specht et al., 1997).

### Example 8

### Therapeutic Antitumor Immunity Induced by DCS Pulsed with CPP1 -TRP2 Peptide

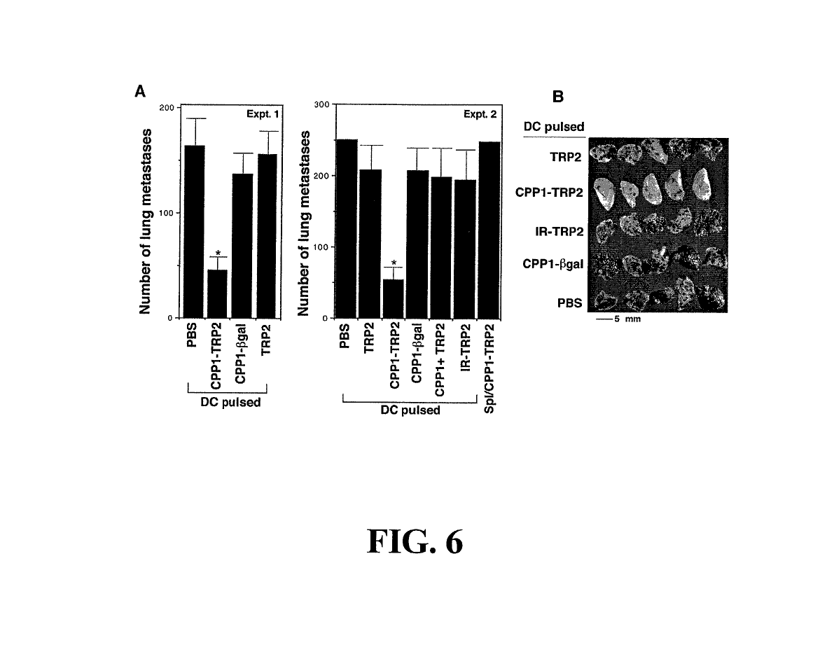

It was next tested whether immunization with DCs loaded with CPP1-TRP2 could generate immune responses strong enough to inhibit the pre-existing B16 tumor. Three days after injection of mice with B16 tumor cells (3×10⁵ cells/mouse), the animals were immunized with a single intravenous injection of DCs loaded with various peptides. Lungs were harvested and lung metastases were counted on day 14. In contrast to the lack of any therapeutic effect in mice immunized with DCs/TRP2 peptide, DCs alone or with control peptides (FIG. 6A), immunization with DCs/CPP1-TRP2 significantly reduced numbers of lung metastases (P<0.01). Interestingly, immunization with spleen cells pulsed with CPP1-TRP2 did not inhibit tumor growth, suggesting that effective immunization requires the combination of mature DCs and CPP1-TRP2. DCs pulsed with the 12-mer CPP1 peptide and the TRP2 peptide, or with IR-TRP2 did not elicit any antitumor immunity against B16 tumor, indicating that physical link between CPP1 and TRP2 is required to carry the TRP2 peptide into DCs and to generate potent antitumor immunity. The superior antitumor responses elicited by DC/CPP1-TRP2 vaccination were reproduced in separate experiments (FIG. 6B).

These results suggest that the weak immunity elicited by DC/TRP2 was not sufficient to inhibit lung metastasis in a 3-day tumor model in a single injection (FIG. 6) or even after multiple vaccinations of DC/TRP2. In contrast, a single vaccination of DCs loaded with CPP-TRP2 peptide resulted in complete elimination of lung metastases and protection of mice from subsequent tumor challenge (FIG. 2), and significant inhibition of pre-existing tumor (FIG. 6). Thus, this is the first demonstration that DC/CPP1-TRP2 immunization generates potent antitumor immunity against poorly immunogenic B16 tumor cells in animal models. Given the fact that many MHC class I-restricted T cell peptides have been identified in cancer cells and infectious viruses, efficient delivery of these T-cell peptides (both class I and/or II peptides) into DCs by cell-penetrating peptides may represent a widely applicable means to enhance immune responses against cancer, and perhaps autoimmune and infectious diseases as well.

### REFERENCES

All patents and publications mentioned in the specification are indicative of the levels of those skilled in the art to which the invention pertains. All patents and publications are herein incorporated by reference to the same extent as if each individual publication was specifically and individually indicated to be incorporated by reference.

### Patents

U.S. Pat. No. 5,440,013

U.S. Pat. No. 5,618,914

U.S. Pat. No. 5,670,155

U.S . Patent 5,446,128

U.S. Pat. No. 5,710,245

U.S. Pat. No. 5,840,833

U.S. Pat. No. 5,859,184

U.S. Pat. No. 5,929,237

U.S. Pat. No. 5,475,085

U.S. Pat. No. 5,672,681

U.S. Pat. No. 5,674,976

U.S. Pat. No. 4,554,101

### Publications

Bakker et al., 1994, J. Exp. Med. 179:1005

Banchereau, J. & Steinman, R. M. Dendritic cells and the control of immunity. Nature 392, 245- 252 (1998).

Beckmann et al., 1992, Eur. J. Cancer 28:322

Berardi et al., Science, 267: 104 (1995)

Bellone, M. et al. Relevance of the tumor antigen in the validation of three vaccination strategies for melanoma. J. Immunol. 165, 2651-2656 (2000).

Berenson, et al., J. Immunol. Meth., 91: 11, 1986) Bishop et al., Blood, vol. 83, No. 2, pp. 610-616 (1994)

Bloom, M. B. et al. Identification of tyrosinase-related protein 2 as a tumor rejection antigen for the B16 melanoma. J.Exp.Med. 185, 453-460 (1997).

Boon, T. & Van der Bruggen , P. Human tumor antigens recognized by T lymphocytes. J.Exp.Med. 183, 725-729 (1996).

Bowne, W. B. et al. Coupling and uncoupling of tumor immunity and autoimmunity. J. Exp. Med. 190, 1717-1722 (1999).

Caron, N. J. et al. Intracellular delivery of a Tat-eGFP fusion protein into muscle cells. Mol Ther 3, 310-318 (2001).

Casares, N. et al. Immunization with a tumor-associated CTL epitope plus a tumor-related or unrelated Thl helper peptide elicits protective CTL immunity. Eur. J. Immunol. 31, 1780-1789 (2001).

Cella, M., Engering, A., Pinet, V., Pieters, J. & Lanzavecchia, A. Inflammatory stimuli induce accumulation of MHC class II complexes on dendritic cells. Nature 388, 782-787 (1997).

Celluzzi, C. M., Mayordomo, J. I., Storkus, W. J., Lotze, M. T. & Falo, L. D., Jr. Peptide-pulsed dendritic cells induce antigen-specific CTL-mediated protective tumor immunity. J. Exp. Med. 183, 283-287 (1996).

Chen Y T, Scanlan M J, Obata Y, Old L J. Identification of human tumor antigens by serological expression cloning (SEREX). In: Principles and Practice of Biologic Therapy of Cancer, 3rd Ed. 2000. Edited by S. A. Rosenberg, Lippincott, Williams & Wilkins, Philadelphia, Pa., p.557-570.

Cohen, P. A. et al. 1994 Cancer Res. 54:1055-1058

Coulie et al., 1994, J. Exp. Med. 180:35

Dallal, R. M. & Lotze, M. T. The dendritic cell and human cancer vaccines. Curr. Opin. Immunol. 12, 583-588 (2000).

Elliott, G. & O'Hare, P. Intercellular trafficking and protein delivery by a herpesvirus structural protein. Cell 88, 223-233 (1997).

Fawell, S. et al. Tat-mediated delivery of heterologous proteins into cells. Proc. Natl. Acad. Sci. USA 91, 664-668 (1994).

Frankel, A. D. & Pabo, C. O. Cellular uptake of the tat protein from human immunodeficiency virus. Cell 55, 1189-1193 (1988).

Gilboa, E. The makings of a tumor rejection antigen. Immunity 11, 263-270 (1999).

Greenberg, P. D. Adoptive T cell therapy of tumors: mechanisms operative in the recognition and elimination of tumor cells. Adv.Immunol. 49, 281-355 (1991).

Hawakami et al., 1994, PNAS 91:3515

Houghton, A. N., Gold, J. S. & Blachere, N. E. Immunity against cancer: lessons learned from melanoma. Curr. Opin. Immunol. 13, 134-140. (2001).

Hung, K. et al. The central role of CD4+ T cells in the antitumor immune iesponse. J.Exp.Med. 188, 2357-2368 (1998).

Iscove, et al., F.J. Exp. Med., 147: 923 (1978)

Johannesson et al., 1999, “Bicyclic tripeptide mimetics with reverse turn inducing properties.” J. Med. Chem. 42:601-608.

Johnson et al., 1993

Kawakami et al., 1994, J. Exp. Med.

Kim, D. T. et al. Introduction of soluble proteins into the MHC class I pathway by conjugation to an HIV tat peptide. J. Immunol. 159, 1666-1668 (1997).

Kyte & Doolittle, 1982

Labeur, M. S. et al. Generation of tumor immunity by bone marrow-derived dendritic cells correlates with dendritic cell maturation stage. J. Immunol. 162, 168-175 (1999).

Lin, Y. Z., Yao, S. Y., Veach, R. A., Torgerson, T. R. & Hawiger, J. Inhibition of nuclear translocation of transcription factor NF-kappa B by a synthetic peptide containing a cell membrane-permeable motif and nuclear localization sequence. J. Biol. Chem. 270, 14255-14258 (1995).

Lindgren, M., Hallbrink, M., Prochiantz, A. & Langel, U. Cell-penetrating peptides. Trends Pharmacol. Sci. 21, 99-103 (2000).

Ludewig, B., Odermatt, B., Ochsenbein, A. F., Zinkernagel, R. M. & Hengartner, H. Role of dendritic cells in the induction and maintenance of autoimmune diseases. Immunol. Rev. 169, 45-54 (1999).

Mehta-Damani, A. et al. 1994 J. Immunol. 153:996-1003 Overwijk, W. W. et al. Vaccination with a recombinant vaccinia virus encoding a “self” antigen induces autoimmune vitiligo and tumor cell destruction in mice: Requirement for CD4+ T lymphocytes. Proc. Natl. Acad. Sci. U S A 96, 2982-2987 (1999).

Paglia, P., Chiodoni, C., Rodolfo, M. & Colombo, M. P. Murine dendritic cells loaded in vitro with soluble protein prime cytotoxic T lymphocytes against tumor antigen in vivo. J. Exp. Med. 183, 317-322 (1996).

Pardoll, D. M. & Topalian, S. L. The role of CD4+ T cell responses in antitumor immunity. Curr. Opin. Immunol. 10, 588-594 (1998).

Parkhurst, M. R. et al. Identification of a shared HLA-A*0201-restricted T-cell epitope from the melanoma antigen tyrosinase-related protein 2 (TRP2). Cancer Res. 58, 4895-4901 (1998).

Parkhurst, M. R. et al. Improved induction of melanoma reactive CTL with peptides from the melanoma antigen gp100 modified at HLA-A0201 binding residues. J.Immunol. 157, 2539-2548 (1996).

Phelan, A., Elliott, G. & O'Hare, P. Intercellular delivery of functional p53 by the herpesvirus protein VP22. Nat. Biotechnol. 16, 440-443 (1998).

Renkvist N et al. A listing of human tumor antigens recognized by T cells. Cancer Immunol Immunother (2001) 50:3-15.

Rojas, M., Donahue, J. P., Tan, Z. & Lin, Y. Z. Genetic engineering of proteins with cell membrane permeability. Nat. Biotechnol. 16, 370-375 (1998).

Romani, et al., J. Exp. Med., 180: 83 (1994)

Rosenberg, S. A. et al. Immunologic and therapeutic evaluation of a synthetic tumor-associated peptide vaccine for the treatment of patients with metastatic melanoma. Nature Med. 4, 321-327 (1998).

Sahin U, Tüeci Ö, Pfreudschuh M. Serological identification of human tumor antigens. Curr. Opin. Immunol. 9:709-716, 1997.

Schreurs, M. W. et al. Dendritic cells break tolerance and induce protective immunity against a melanocyte differentiation antigen in an autologous melanoma model. Cancer Res. 60, 6995-7001 (2000).

Schuler, G. & Steinman, R. M. Dendritic cells as adjuvants for immune-mediated resistance to tumors. J. Exp. Med. 186, 1183-1187. (1997).

Schwarze, S. R., Ho, A., Vocero-Akbani, A. & Dowdy, S. F. In vivo protein transduction: delivery of a biologically active protein into the mouse. Science 285, 1569-1572 (1999).

Slansky, J. E. et al. Enhanced antigen-specific antitumor immunity with altered peptide ligands that stabilize the MHC-peptide-TCR complex. Immunity 13, 529-538 (2000).

Specht, J. M. et al. Dendritic cells retrovirally transduced with a model antigen gene are therapeutically effective against established pulmonary metastases. J.Exp.Med. 186, 1213-1221 (1997).

Srivastava, P. K. Immunotherapy of human cancer: lessons from mice. Nat. Immunol. 1, 363-366 (2000).

Steinman, et al., Ann. Rev. Immunol. 9: 271 (1991)

Suhrbier, A. et al. Peptide epitope induced apoptosis of human cytotoxic T lymphocytes. Implications for peripheral T cell deletion and peptide vaccination. J. ImmunoL. 150, 2169-2178 (1993).

Thurner, B. et al. Vaccination with mage-3A1 peptide-pulsed mature, monocyte-derived dendritic cells expands specific cytotoxic T cells and induces regression of some metastases in advanced stage IV melanoma. J. Exp. Med. 190, 1669-1678 (1999).

Toes, R. E., Ossendorp, F., Offringa, R. & Melief, C. J. CD4 T cells and their role in antitumor immune responses. J.Exp.Med. 189, 753-756 (1999).

Vita et al., 1998, “Novel miniproteins engineered by the transfer of active sites to small natural scaffolds.” Biopolymers 47:93-100.

Wang, R.-F., Appella, E., Kawakami, Y., Kang, X. & Rosenberg, S. A. Identification of TRP-2 as a human tumor antigen recognized by cytotoxic T lymphocytes. J Exp Med 184, 2207-2216 (1996).

Wang, R.-F., Nature Biotechnology, Vol. 20, 149-156, 2002.

Wang, R.-F. The role of MHC class II-restricted tumor antigens and CD4+ T cells in antitumor immunity. Trends in Immunology 22, 269-276 (2001).

Wang, R.-F. & Rosenberg, S. A. Human tumor antigens for cancer vaccine development. Immunol. Rev. 170, 85-100 (1999).

Weisshoff et al., 1999, “Mimicry of beta II'-turns of proteins in cyclic pentapeptides with one and without D-amino acids.” Eur. J. Biochem. 259:776-788.

Winzler, C. et al. Maturation stages of mouse dendritic cells in growth factor-dependent long-term cultures. J.Exp.Med.185, 317-328 (1997).

Young, J. W. & Inaba, K. Dendritic cells as adjuvants for class I major histocompatibility complex-restricted antitumor immunity. J.Exp.Med. 183, 7-11 (1996).

Zeh, H. J., 3rd, Perry-Lalley, D., Dudley, M. E., Rosenberg, S. A. & Yang, J. C. High avidity CTLs for two self-antigens demonstrate superior in vitro and in vivo antitumor efficacy. J Immunol 162, 989-994 (1999).

I claim:

1. A composition comprising an immune effector cell and a cell penetrating peptide, wherein said cell penetrating peptide is associated with an antigen.

2. The composition of claim 1, wherein the antigen is a tumor rejection antigen or tumor associated antigen.

3. The composition of claim 1, wherein the antigen is a molecule comprising multiple T-cell peptides.

4. The composition of claim 3, wherein the multiple T-cell peptides are from either the same tumor antigen or different tumor antigens.

5. The composition of claim 1, wherein the antigen comprises at least one MHC class I-restricted peptide, at least one MHC class II-restricted peptide, or at least one MHC class I-restricted peptide and at least one MHC class II-restricted peptide.

6. The composition of claim 1, wherein the immune effector cell is a mature dendritic cell, a B cell, a macrophage, or a fibroblast.

7. The composition of claim 1, wherein the immune effector cell is a mature dendritic cell or a B cell.

8. The composition of claim 1, wherein the immune effector cell is a mature dendritic cell.

9. The composition of claim 1, wherein the antigen is a tumor antigen.

10. The composition of claim 9, wherein the tumor antigen is a peptide.

11. The composition of claim 9, wherein the tumor antigen is TRP2.

12. The composition of claim 9, wherein the tumor antigen is one from Table 1, Table 2, Table 3, Table 4, or Table 5.

13. The composition of claim 1, wherein the cell penetrating peptide is CPP1, ANTP, Signal-peptide I, Signal-peptide II, PRES, Transportan, Amphiphilic model peptide, HSV VP22, peptide carrier, or CL22.

14. The composition of claim 1, wherein the cell penetrating peptide is CPP1.

15. The composition of claim 1, wherein the association of the cell penetration peptide with the antigen is a covalent bond.

16. The composition of claim 1, wherein the antigen is housed within a vesicle in said immune system cell.

17. The composition of claim 16, wherein the vesicle is an endosome.

18. A composition comprising an immune effector cell and a cell penetrating peptide, wherein said cell penetrating peptide is associated with an antibody.

19. A vaccine comprising:
an immune effector cell and a cell penetrating peptide, wherein said cell penetrating peptide is associated with an antigen; and
a pharmaceutically acceptable carrier.

20. The vaccine of claim 19, wherein the immune effector cell is a mature dendritic cell, a B cell, a macrophage, or a fibroblast.

21. The vaccine of claim 19, wherein the immune effector cell is a mature dendritic cell or a B cell.

22. The vaccine of claim 19, wherein the immune effector cell is a mature dendritic cell.

23. A method of enhancing immunity in an animal to a disease, comprising the step of administering to the animal a mature dendritic cell, wherein the cell comprises a cell penetrating peptide associated with an antigen to said disease, wherein following said administration, said animal is protected from said disease.

24. The method of claim 23, wherein said animal comprises both CD4+ and CD8+ T cells.

25. The method of claim 23, wherein said dendritic cell is administered to the animal by injection.

26. The method of claim 25, wherein said injection is intravenously, intraperitoneally, or subcutaneously.

27. The method of claim 23, wherein the animal is a mammal.

28. The method of claim 27, wherein the mammal is a human.

29. A method of immunizing an animal, comprising administering the vaccine of claim 18 at least once to said animal.

30. A method of treating a disease in an animal, comprising the step of administering to the animal:
an immune effector cell comprising a cell-penetrating peptide associated with an antigen for said disease; and
a pharmaceutically acceptable carrier.

31. The method of claim 30, wherein the immune effector cell is a mature dendritic cell, a B cell, a macrophage, or a fibroblast.

32. The method of claim 30, wherein the immune effector cell is a mature dendritic cell or a B cell.

33. The method of claim 30, wherein the immune effector cell is a mature dendritic cell.

34. The method of claim 30, wherein the cell penetrating peptide is CPP1, HIV Tat, VP22, MTS, or fibroblast growth factor.

35. The method of claim 30, wherein the cell-penetrating peptide is CPP1.

36. The method of claim 30, wherein the disease is cancer and wherein the antigen is a tumor antigen.

37. The method of claim 36, wherein the tumor antigen is TRP2.

38. The method of claim 36, wherein the tumor antigen is one from Table 1, Table 2, Table 3, Table 4, or Table 5.

39. The method of claim 30, wherein the animal is further subjected to a cancer treatment, wherein the treatment is surgery, radiation, chemotherapy, or gene therapy.

40. The method of claim 39 wherein the administration of the dendritic cell is prior to the cancer treatment.

41. The method of claim 39, wherein the administration of the dendritic cell is subsequent to the cancer treatment.

42. The method of claim 39, wherein the administration of the dendritic cell is concurrent with the cancer treatment.

43. A method of preparing a composition for a disease, comprising:
providing an immune effector cell;
providing a cell penetrating peptide associated with an antigen for said disease; and
introducing the cell penetrating peptide associated with the antigen to the immune effector cell, wherein said antigen enters into the cell.

44. The method of claim 43, wherein the immune effector cell is a mature dendritic cell, B cell, a macrophage, or a fibroblast.

45. The method of claim 43, wherein the immune effector cell is a mature dendritic cell.

46. The method of claim 43, wherein the antigen is a tumor antigen, autoantigen, or viral antigen.


SEQUENCE LISTING (14 sequences)

SEQ ID NO: 1 - protein, 12 aa, Artificial Sequence
  Synthetic Peptide
AAVLLPVLLAAP

SEQ ID NO: 2 - protein, 9 aa, Human
SVYDFFVWL

SEQ ID NO: 3 - protein, 16 aa, Drosophila
RQIKIWFQNRRMKWKK

SEQ ID NO: 4 - protein, 11 aa, Human Immunodeficiency Virus
YGRKKRRQRRR

SEQ ID NO: 5 - protein, 11 aa, Artificial Sequence
  Synthetic Peptide
YARAAARQARA

SEQ ID NO: 6 - protein, 11 aa, Artificial Sequence
  Synthetic Peptide
YARAARRAARR

SEQ ID NO: 7 - protein, 16 aa, human
AAVALLPAVLLALLAP

SEQ ID NO: 8 - protein, 27 aa, Artificial Sequence
  Synthetic Peptide
GALFLGWLGAAGSTMGAWSQPKKKRKV

SEQ ID NO: 9 - protein, 12 aa, Hepatitis B virus
PLSSIFSRIGDP

SEQ ID NO: 10 - protein, 26 aa, Artificial Sequence
  Synthetic Peptide
GWTLNSAGYLLKINLKALAALAKKIL

SEQ ID NO: 11 - protein, 18 aa, Artificial Sequence
  Synthetic Peptide
KLALKLALKALKAALKLA

SEQ ID NO: 12 - protein, 34 aa, Herpes simplex virus 1
DAATATRGRSAASRPTERPRAPARSASRPRRPVE

SEQ ID NO: 13 - protein, 21 aa, Artificial Sequence
  Synthetic Peptide
KETWWETWWTEWSQPKKKRKV

SEQ ID NO: 14 - protein, 35 aa, Artificial Sequence
  Synthetic Peptide
KKKKKKGGFLGFWRGENGRKTRSAYERMCNILKGK